(19) United States
(12) Patent Application Publication
(10) Pub. No.: US 2019/0298304 A1
(43) Pub. Date: Oct. 3, 2019

(54) MEDICAL DIAGNOSIS APPARATUS, MEDICAL IMAGE PROCESSING APPARATUS, AND IMAGE PROCESSING METHOD

(71) Applicant: Canon Medical Systems Corporation, Otawara-shi (JP)

(72) Inventors: Yu Igarashi, Kawasaki (JP); Masaki Watanabe, Utsunomiya (JP); Yasunori Honjo, Kawasaki (JP); Tetsuya Kawagishi, Nasushiobara (JP)

(73) Assignee: Canon Medical Systems Corporation, Otawara-shi (JP)

(21) Appl. No.: 16/369,783

(22) Filed: Mar. 29, 2019

(30) Foreign Application Priority Data
Mar. 30, 2018 (JP) 2018-068884
Mar. 28, 2019 (JP) 2019-062812

(51) Int. Cl. A61B 8/14; A61B 8/06; A61B 8/08

(52) U.S. Cl. CPC A61B 8/14; A61B 8/4444; A61B 8/481; A61B 8/06

(57) ABSTRACT

An ultrasound diagnosis apparatus according to an embodiment includes an image generating unit, a specifying unit, and an obtaining unit. The image generating unit is configured to generate images in a time series on the basis of a result of a scan performed on a scan region. The specifying unit is configured to specify the position of a moving member included in the scan region, with respect to each of the images in the time series. The obtaining unit is configured to obtain movement information of the moving member on the basis of the positions of the moving member and to obtain a moment of first or higher order related to the movement information of the moving member, with respect to at least a part of the scan region.

### CROSS-REFERENCE TO RELATED APPLICATIONS

This application is based upon and claims the benefit of priority from Japanese Patent Application No. 2018-068884, filed on Mar. 30, 2018 and Japanese Patent Application No. 2019-062812, filed on Mar. 28, 2019; the entire contents of which are incorporated herein by reference.

### FIELD

Embodiments described herein relate generally to a medical diagnosis apparatus, a medical image processing apparatus, and an image processing method.

### BACKGROUND

Conventionally, an ultrasound diagnosis apparatus is configured to render dynamics of a blood flow in an image, by using an imaging method that uses the Doppler effect. For example, a technique is provided by which the velocity of a moving member or a statistical value based on the velocity thereof is calculated and rendered in an image by using the Doppler effect, so as to assist viewers in distinguishing arteries and veins from each other. However, strictly speaking, this imaging method calculates only a velocity component in the direction of a beam transmitted and received by the ultrasound probe. Thus, this imaging method does not necessarily acquire an accurate velocity component in the actual direction of the blood flow.

### BRIEF DESCRIPTION OF THE DRAWINGS

FIG. 1 is a block diagram illustrating an exemplary configuration of an ultrasound diagnosis apparatus according to a first embodiment;

FIG. 2 is a drawing for explaining a process performed by a specifying function according to the first embodiment;

FIG. 3 is a drawing for explaining a process performed by a setting function according to the first embodiment;

FIG. 4 is a drawing for explaining a process performed by a first calculating function according to the first embodiment;

FIG. 5 is a drawing for explaining a process performed by a second calculating function according to the first embodiment;

FIG. 6 is another drawing for explaining the process performed by the second calculating function according to the first embodiment;

FIG. 7A is yet another drawing for explaining the process performed by the second calculating function according to the first embodiment;

FIG. 7B is yet another drawing for explaining the process performed by the second calculating function according to the first embodiment;

FIG. 8A is a drawing for explaining a process performed by a display controlling function according to the first embodiment;

FIG. 8B is another drawing for explaining the process performed by the display controlling function according to the first embodiment;

FIG. 9 is a flowchart for explaining a processing procedure performed by the ultrasound diagnosis apparatus according to the first embodiment;

FIG. 10 is a drawing for explaining a process performed by a second calculating function according to a modification example of the first embodiment;

FIG. 11 is a drawing for explaining a process performed by a second calculating function according to another modification example of the first embodiment;

FIG. 12 is a drawing for explaining a process performed by a second calculating function according to yet another modification example of the first embodiment;

FIG. 13 is a drawing for explaining a process performed by an ultrasound diagnosis apparatus according to a second embodiment;

FIG. 14 is a drawing for explaining a process performed by an ultrasound diagnosis apparatus according to a third embodiment;

FIG. 15 is another drawing for explaining the process performed by the ultrasound diagnosis apparatus according to the third embodiment;

FIG. 16 is yet another drawing for explaining the process performed by the ultrasound diagnosis apparatus according to the third embodiment;

FIG. 17 is a drawing for explaining a process performed by an ultrasound diagnosis apparatus according to a fourth embodiment;

FIG. 18 is a drawing for explaining a process performed by an ultrasound diagnosis apparatus according to a fifth embodiment; and

FIG. 19 is a drawing for explaining a process performed by an ultrasound diagnosis apparatus according to another embodiment.

### DETAILED DESCRIPTION

It is an object of the present disclosure to provide a medical diagnosis apparatus, a medical image processing apparatus, and an image processing method that are capable of accurately evaluating dynamics of blood flows.

A medical diagnosis apparatus according to an embodiment includes processing circuitry. The processing circuitry is configured to generate images in a time series on the basis of a result of a scan performed on a scan region. The processing circuitry is configured to specify a position of a moving member included in the scan region, with respect to each of the images in the time series. The processing circuitry is configured to obtain movement information of the moving member on the basis of the positions of the moving member and to obtain a moment of first or higher order related to the movement information of the moving member, with respect to at least a part of the scan region.

Exemplary embodiments of a medical diagnosis apparatus, a medical image processing apparatus, and an image processing method will be explained below, with reference to the accompanying drawings. The embodiments described below are merely examples, and possible embodiments are not limited to the embodiments described below. Further, it is possible, in principle, to similarly apply the description of each of the embodiments to any other embodiment.

### First Embodiment

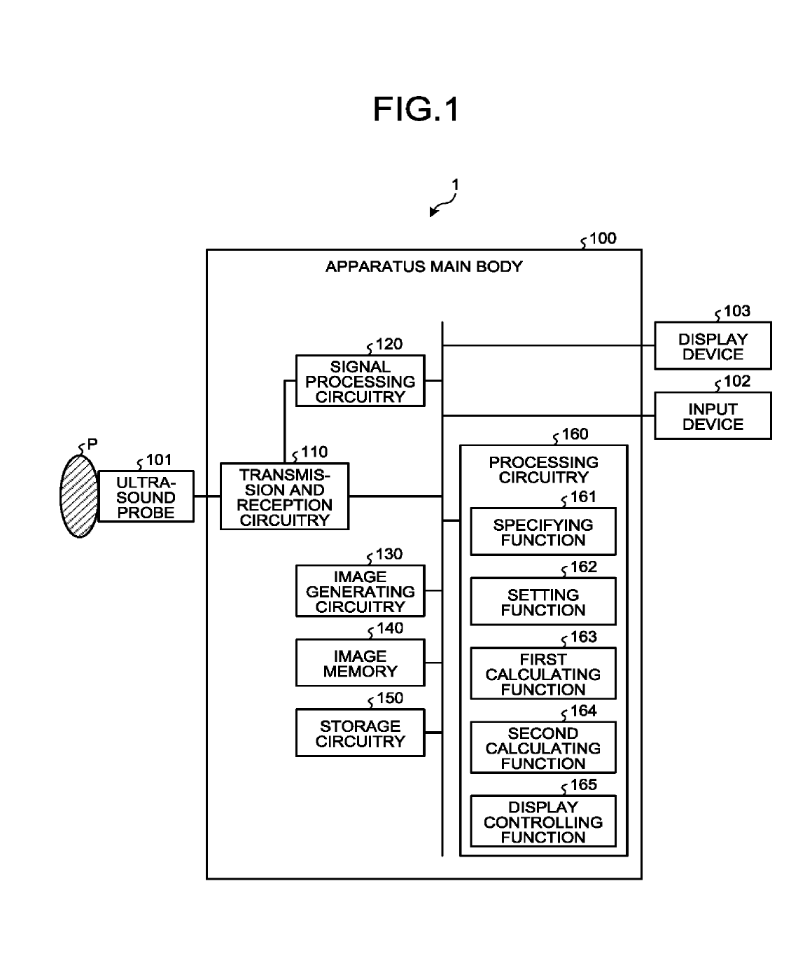

FIG. 1 is a block diagram illustrating an exemplary configuration of an ultrasound diagnosis apparatus 1 according to a first embodiment. As illustrated in FIG. 1, the ultrasound diagnosis apparatus 1 according to the first embodiment includes an apparatus main body 100, an ultrasound probe 101, an input device 102, and a display device 103. The ultrasound probe 101, the input device 102, and the display device 103 are connected to the apparatus main body 100. An examined subject (hereinafter “patient”) P is not included in the configuration of the ultrasound diagnosis apparatus 1. The ultrasound diagnosis apparatus 1 is an example of the medical diagnosis apparatus.

The ultrasound probe 101 includes a plurality of transducer elements (e.g., piezoelectric transducer elements). The plurality of transducer elements are configured to generate ultrasound waves on the basis of a drive signal supplied thereto from transmission and reception circuitry 110 (explained later) included in the apparatus main body 100. Further, the plurality of transducer elements included in the ultrasound probe 101 are configured to receive reflected waves from the patient P and to convert the received reflected waves into electric signals. Further, the ultrasound probe 101 includes a matching layer provided for the transducer elements, as well as a backing member or the like that prevents the ultrasound waves from propagating rearward from the transducer elements.

When an ultrasound wave is transmitted from the ultrasound probe 101 to the patient P, the transmitted ultrasound wave is repeatedly reflected on a surface of discontinuity of acoustic impedances at a tissue in the body of the patient P and is received as a reflected-wave signal (an echo signal) by each of the plurality of transducer elements included in the ultrasound probe 101. The amplitude of the received reflected-wave signal is dependent on the difference between the acoustic impedances on the surface of discontinuity on which the ultrasound wave is reflected. When a transmitted ultrasound pulse is reflected on the surface of a moving blood flow, a cardiac wall, or the like, the reflected-wave signal is, due to the Doppler effect, subject to a frequency shift, depending on a velocity component of the moving members with respect to the ultrasound wave transmission direction.

The first embodiment is applicable to any of the following situations: the situation where the ultrasound probe 101 illustrated in FIG. 1 is a one-dimensional ultrasound probe in which the plurality of piezoelectric transducer elements are arranged in a row; the situation where the ultrasound probe 101 is a one-dimensional ultrasound probe in which the plurality of piezoelectric transducer elements arranged in a row are mechanically swung; and the situation where the ultrasound probe 101 is a two-dimensional ultrasound probe in which the plurality of piezoelectric transducer elements are two-dimensionally arranged in a grid formation.

The input device 102 includes a mouse, a keyboard, a button, a panel switch, a touch command screen, a foot switch, a trackball, a joystick, and/or the like and is configured to receive various types of setting requests from an operator of the ultrasound diagnosis apparatus 1 and to transfer the received various types of setting requests to the apparatus main body 100.

The display device 103 is configured to display a Graphical User Interface (GUI) used by the operator of the ultrasound diagnosis apparatus 1 to input the various types of setting requests through the input device 102 and to display ultrasound image data generated by the apparatus main body 100 and the like.

The apparatus main body 100 is an apparatus configured to generate the ultrasound image data on the basis of the reflected-wave signals received by the ultrasound probe 101. As illustrated in FIG. 1, the apparatus main body 100 includes the transmission and reception circuitry 110, signal processing circuitry 120, image generating circuitry 130, an image memory 140, storage circuitry 150, and processing circuitry 160. The transmission and reception circuitry 110, the signal processing circuitry 120, the image generating circuitry 130, the image memory 140, the storage circuitry 150, and the processing circuitry 160 are connected so as to be able to communicate with one another.

The transmission and reception circuitry 110 includes a pulse generator, a transmission delay unit, a pulser, and the like and is configured to supply the drive signal to the ultrasound probe 101. The pulse generator is configured to repeatedly generate a rate pulse used for forming a transmission ultrasound wave at a predetermined rate frequency. Further, the transmission delay unit is configured to apply a delay period that is required to converge the ultrasound wave generated by the ultrasound probe 101 into the form of a beam and to determine transmission directionality and that corresponds to each of the piezoelectric transducer elements, to each of the rate pulses generated by the pulse generator. Further, the pulser is configured to apply the drive signal (a drive pulse) to the ultrasound probe 101 with timing based on the rate pulses. In other words, by varying the delay periods applied to the rate pulses, the transmission delay unit is able to arbitrarily adjust the transmission directions of the ultrasound waves transmitted from the surfaces of the piezoelectric transducer elements.

In this situation, the transmission and reception circuitry 110 has a function that is able to instantly change the transmission frequency, the transmission drive voltage, and the like, for the purpose of executing a predetermined scan sequence on the basis of an instruction from the processing circuitry 160 (explained later). In particular, the function to change the transmission drive voltage is realized by using a linear-amplifier-type transmission circuitry of which the value can be instantly switched or by using a mechanism configured to electrically switch between a plurality of power source units.

Further, the transmission and reception circuitry 110 includes a pre-amplifier, an Analog/Digital (A/D) converter, a reception delay unit, an adder, and the like and is configured to generate reflected-wave data by performing various types of processes on the reflected-wave signals received by the ultrasound probe 101. The pre-amplifier is configured to amplify the reflected-wave signals for each of the channels. The A/D converter is configured to perform an Analog/Digital (A/D) conversion process on the amplified reflected-wave signals. The reception delay unit is configured to apply a delay period required to determine reception directionality, to the result of the A/D conversion. The adder is configured to generate the reflected-wave data by performing an adding process on the reflected-wave signals processed by the reception delay unit. As a result of the adding process performed by the adder, reflected components from the direction corresponding to the reception directionality of the reflected-wave signals are emphasized, so that a comprehensive beam used in the ultrasound transmission and reception is formed on the basis of the reception directionality and the transmission directionality.

When a two-dimensional region of the patient P is to be scanned, the transmission and reception circuitry 110 is configured to cause an ultrasound beam to be transmitted in a two-dimensional direction from the ultrasound probe 101. Further, the transmission and reception circuitry 110 is configured to generate two-dimensional reflected-wave data from the reflected-wave signals received by the ultrasound probe 101. In contrast, when a three-dimensional region of the patient P is to be scanned, the transmission and reception circuitry 110 is configured to cause an ultrasound beam to be transmitted in a three-dimensional direction from the ultrasound probe 101. Further, the transmission and reception circuitry 110 is configured to generate three-dimensional reflected-wave data from the reflected-wave signals received by the ultrasound probe 101.

The signal processing circuitry 120 is configured to generate data (B-mode data) in which the signal intensity at each of the sampling points is expressed by a level of brightness, by performing, for example, a logarithmic amplification process, an envelope detection process, and/or the like on the reflected-wave data received from the transmission and reception circuitry 110. The B-ode data generated by the signal processing circuitry 120 is output to the image generating circuitry 130.

Further, the signal processing circuitry 120 is capable of varying the frequency band to be rendered in images, by varying the detected frequency through a filtering process. By using this function of the signal processing circuitry 120, it is possible to execute a contrast enhanced echo method such as a Contrast Harmonic Imaging (CHI) process, for example. In other words, from the reflected-wave data of the patient P into whom a contrast agent has been injected, the signal processing circuitry 120 is capable of separating reflected-wave data (a harmonic component or a subharmonic component) reflected by the contrast agent represented by microbubbles and reflected-wave data (a fundamental wave component) reflected by tissues on the inside of the patient P. Accordingly, the signal processing circuitry 120 is able to extract either the harmonic component or the subharmonic component from the reflected-wave data of the patient P and to generate B-mode data used for generating contrast enhanced image data (harmonic image data). The B-mode data used for generating the contrast enhanced image data is data in which the signal intensities of the reflected waves that were reflected by the contrast agent are expressed with levels of brightness. Further, the signal processing circuitry 120 is also able to extract the fundamental wave component from the reflected-wave data of the patient P and to generate B-mode data used for generating tissue image data (fundamental image data).

When performing the CHI process, the signal processing circuitry 120 is capable of extracting the harmonic component (a higher harmonic wave component) by using a method different from the abovementioned method that employs the filtering process. During the harmonic imaging process, an imaging method may be implemented such as an Amplitude Modulation (AM) method; a Phase Modulation (PM) method; or an AMPM method in which the AM method and the PM method are combined together. According to the AM method, the PM method, and the AMPM method, ultrasound wave transmission sessions having mutually-different amplitude levels and/or mutually-different phases are performed multiple times (at multiple different rates) on mutually the same scanning line. As a result, the transmission and reception circuitry 110 generates and outputs a plurality of pieces of reflected-wave data for each of the scanning lines. Further, the signal processing circuitry 120 extracts the harmonic component by performing an adding/subtracting process corresponding to the modulation method, on the plurality of pieces of reflected-wave data corresponding to the scanning lines. After that, the signal processing circuitry 120 generates B-mode data by performing the envelope detecting process or the like on the reflected-wave data of the harmonic component.

For example, when the PM method is implemented, according to a scan sequence set by the processing circuitry 160, the transmission and reception circuitry 110 causes ultrasound waves having opposite phase polarities and mutually the same amplitude levels (e.g., −1 and 1) to be transmitted twice for each of the scanning lines. Further, the transmission and reception circuitry 110 generates a piece of reflected-wave data resulting from the transmission corresponding to “−1” and another piece of reflected-wave data resulting from the transmission corresponding to “1”, so that the signal processing circuitry 120 adds the two pieces of reflected-wave data together. As a result, a signal is generated from which the fundamental wave component has been eliminated and in which a second harmonic component primarily remains. Further, the signal processing circuitry 120 generates CHI B-mode data (B-mode data used for generating contrast enhanced image data) by performing an envelope detecting process or the like on the generated signal. The CHI B-mode data is data in which signal intensities of the reflected waves reflected by the contrast agent are expressed by levels of brightness. Further, when the PM method is implemented during a CHI process, the signal processing circuitry 120 is capable of generating B-mode data used for generating tissue image data, by performing, for example, a filtering process on the reflected-wave data resulting from the transmission corresponding to “1”.

Further, the signal processing circuitry 120 is configured to generate, for example, data (Doppler data) obtained by extracting movement information based on the Doppler effect exerted on moving members at sampling points in a scan region, from the reflected-wave data received from the transmission and reception circuitry 110. More specifically, the signal processing circuitry 120 generates the data (the Doppler data) obtained by extracting moving member information such as average velocity, dispersion, power, and the like with respect to multiple points, by performing a frequency analysis to obtain velocity information from received reflected-wave data and extracting a blood flow, a tissue, and a contrast agent echo component influenced by the Doppler effect. In this situation, the moving members may be, for example, blood flows, tissues such as the cardiac wall, a contrast agent, and/or the like. The movement information (the blood flow information) obtained by the signal processing circuitry 120 is forwarded to the image generating circuitry 130 so as to be displayed in color on the display device 103 as an average velocity image, a dispersion image, and/or a power image, or an image combining together any of these images.

The image generating circuitry 130 is configured to generate ultrasound image data from the data generated by the signal processing circuitry 120. The image generating circuitry 130 is configured to generate the B-mode image data in which the intensities of the reflected-waves are expressed with levels of brightness, from the B-mode data generated by the signal processing circuitry 120. Further, the image generating circuitry 130 is configured to generate the contrast enhanced image data (the harmonic image data) on the basis of the harmonic component or the subharmonic component extracted from the reflected-wave data of the patient P. Further, on the basis of the fundamental wave component extracted from the reflected-wave data of the patient P, the image generating circuitry 130 is configured to generate the tissue image data (the fundamental image data). Further, the image generating circuitry 130 is configured to generate the Doppler image data expressing the moving member information, from the Doppler data generated by the signal processing circuitry 120. The Doppler image data may be velocity image data, dispersion image data, a power image data, or image data combining together any of these types of image data. The image generating circuitry 130 is an example of the image generating unit configured to generate images in a time series on the basis of a result of a scan performed on the scan region.

In this situation, generally speaking, the image generating circuitry 130 converts (by performing a scan convert process) a scanning line signal sequence from an ultrasound scan into a scanning line signal sequence in a video format used by, for example, television and generates display-purpose ultrasound image data. More specifically, the image generating circuitry 130 generates the display-purpose ultrasound image data by performing a coordinate transformation process compliant with the ultrasound scan mode used by the ultrasound probe 101. Further, as various types of image processing processes besides the scan convert process, the image generating circuitry 130 performs, for example, an image processing process (a smoothing process) to re-generate a brightness average value image, an image processing process (an edge enhancement process) that uses a differential filter inside an image, or the like, by using a plurality of image frames resulting from the scan convert process. Also, the image generating circuitry 130 combines additional information (e.g., text information of various types of parameters, scale graduations, body marks) with the ultrasound image data.

In other words, the B-mode data and the Doppler data are each ultrasound image data before the scan convert process. The data generated by the image generating circuitry 130 is display-purpose ultrasound image data after the scan convert process. When the signal processing circuitry 120 has generated three-dimensional data (three-dimensional B-mode data and three-dimensional Doppler data), the image generating circuitry 130 is configured to generate volume data by performing a coordinate transformation process thereon in accordance with the ultrasound scan mode used by the ultrasound probe 101. After that, the image generating circuitry 130 is configured to generate display-purpose two-dimensional data by performing various types of rendering processes on the volume data.

The image memory 140 is a memory configured to store therein the display-purpose image data generated by the image generating circuitry 130. Further, the image memory 140 is also capable of storing therein any of the data generated by the signal processing circuitry 120. For example, the operator is able to invoke any of the B-mode data and the Doppler data stored in the image memory 140 after a diagnosing process. The invoked B-mode data and Doppler data can serve as the display-purpose ultrasound image data after being routed through the image generating circuitry 130.

The storage circuitry 150 is configured to store therein control programs for performing ultrasound transmissions and receptions, image processing processes, and display processes as well as various types of data such as diagnosis information (e.g., patients' IDs, medical doctors' observations), diagnosis protocols, various types of body marks, and the like. Further, the storage circuitry 150 may also be used, as necessary, for saving therein any of the image data stored in the image memory 140, and the like. Further, the data stored in the storage circuitry 150 may be transferred to an external apparatus via an interface (not illustrated).

The processing circuitry 160 is configured to control overall processes performed by the ultrasound diagnosis apparatus 1. More specifically, the processing circuitry 160 is configured to control processes performed by the transmission and reception circuitry 110, the signal processing circuitry 120, and the image generating circuitry 130, on the basis of the various types of setting requests input thereto by the operator via the input device 102 and the various types of control programs and the various types of data read from the storage circuitry 150. Further, the processing circuitry 160 is configured to exercise control so that the display device 103 displays the display-purpose ultrasound image data stored in the image memory 140.

Further, as illustrated in FIG. 1, the processing circuitry 160 is configured to perform a specifying function 161, a setting function 162, a first calculating function 163, a second calculating function 164, and a display controlling function 165. In this situation for example, processing functions executed by the constituent elements of the processing circuitry 160 illustrated in FIG. 1, namely, the specifying function 161, the setting function 162, the first calculating function 163, the second calculating function 164, and the display controlling function 165, are each recorded in a storage device (e.g., the storage circuitry 150) of the ultrasound diagnosis apparatus 1 in the form of a computer-executable program. The processing circuitry 160 is a processor configured to realize the functions corresponding to the programs by reading and executing the programs from the storage device. In other words, the processing circuitry 160 that has read the programs has the functions illustrated within the processing circuitry 160 in FIG. 1. Processing functions executed by the specifying function 161, the setting function 162, the first calculating function 163, the second calculating function 164, and the display controlling function 165 will be explained later.

In FIG. 1, the example is explained in which the single piece of processing circuitry (i.e., the processing circuitry 160) realizes the processing functions executed by the specifying function 161, the setting function 162, the first calculating function 163, the second calculating function 164, and the display controlling function 165. However, another arrangement is also acceptable in which the processing circuitry is structured by combining together a plurality of independent processors, so that the functions are realized as a result of the processors executing the programs.

A basic configuration of the ultrasound diagnosis apparatus 1 according to the first embodiment has thus been explained. The ultrasound diagnosis apparatus 1 according to the first embodiment configured as described above makes it possible to accurately evaluate dynamics of blood flows, by performing the processes described below. For example, the ultrasound diagnosis apparatus 1 is able to accurately evaluate the dynamics of the blood flows, by tracking each of microbubbles used as a contrast agent while implementing a contrast enhanced echo method.

In the embodiments described below, an example will be explained in which the flow of a contrast agent is rendered by performing a real-time process on ultrasound image data taken by injecting the contrast agent to the patient P. However, possible embodiments are not limited to this example. For instance, it is also possible to perform the process in a retroactive manner on ultrasound imaged data (or reflected-wave data) that has already been taken. In the following sections, the contrast agent may simply be referred to as “bubbles”.

The specifying function 161 is configured to specify the positions of moving members included in a scan region, with respect to each of the images in a time series. In this situation, the moving members may be, for example, bubbles.

For instance, the specifying function 161 specifies the positions of the contrast agent bubbles in a first medical image corresponding to a first temporal phase and in a second medical image corresponding to a second temporal phase. In one example, the specifying function 161 corrects movements of a tissue in the first medical image and in the second medical image and specifies the positions of the contrast agent bubbles in each of the corrected first and second medical images. After that, the specifying function 161 eliminates a harmonic component based on a fixed position in each of the first and the second medical images and specifies the positions of the contrast agent bubbles by using the harmonic component based on the contrast agent bubbles in each of the first and the second medical images resulting from the harmonic component eliminating process. The specifying function 161 is an example of the specifying unit.

First, the specifying function 161 is configured to perform the process of correcting the movements of the tissue, in the contrast enhanced image data taken in a real-time manner. In this situation, the movements of the tissue subject to the correcting process are, for example, overall positional shifting of the image caused by the movements (body movements) of a parenchyma of the patient P and shifting (a sway) of the ultrasound probe 101. In other words, when there is such positional shifting, the positions of the bubbles rendered in the contrast enhanced image data include the movements of the patient and the shifting of the ultrasound probe 101. For this reason, the movements of the tissue in the contrast enhanced image data are corrected.

For example, the specifying function 161 reads, from the image memory 140, a piece of tissue image data in a current frame (which may be referred to as an “n-th frame”) and another piece of tissue image data in an (n−1)-th frame. In this situation, the pieces of tissue image data are each ultrasound image data (B-mode image data) generated on the basis of a fundamental wave component separated from reflected-wave data by performing a filtering process. After that, the specifying function 161 calculates a shift amount between the piece of tissue image data in the n-th frame and the piece of tissue image data in the (n−1)-th frame, by performing a pattern matching process while implementing a cross correlation method on the piece of tissue image data in the n-th frame and the piece of tissue image data in the (n−1)-th frame. Subsequently, by using the calculated shift amount, the specifying function 161 calculates a correction amount used for arranging the coordinate system of the piece of tissue image data in the n-th frame to coincide with the coordinate system of the piece of tissue image data in the (n−1)-th frame. After that, the specifying function 161 corrects the coordinate system of the piece of contrast enhanced image data in the n-th frame, by using the calculated correction amount. In this situation n denotes a natural number.

In this manner, the specifying function 161 performs the correcting process to eliminate the movement (the positional shift) of the tissue between the (n−1)-th frame and the n-th frame, from the piece of contrast enhanced image data in the n-th frame. Accordingly, the specifying function 161 corrects the movement of the tissue in the pieces of contrast enhanced image data in the frames consecutively taken in a real-time manner, while using the position of the tissue in the first frame as a reference.

In the explanation above, the example is explained in which the process is performed by using the tissue image data based on the fundamental wave component obtained by the filtering process; however, possible embodiments are not limited to this example. For instance, when contrast enhanced image data is generated by implementing the PM method, it is also acceptable to use tissue image data generated from reflected-wave data obtained by implementing the PM method. For example, according to the PM method, when the reflected-wave data is obtained by transmitting an ultrasound wave twice at the levels of −1 and 1, B-mode image data obtained from the reflected-wave data resulting from the transmission at the level “1” may be used as the tissue image data described above. Alternatively, it is also acceptable to use, as the tissue image data described above, B-mode image data acquired from a subtraction signal obtained by subtracting the reflected-wave data of the transmission at the level “−1” from the reflected-wave data of the transmission at the level “−1”.

Further, in the explanation above, the example is explained in which the correcting process is performed by using the position of the tissue in the first frame as a reference; however, possible embodiments are not limited to this example. For instance, it is also acceptable to correct the position of the tissue in another frame, by using the position of the tissue in the n-th frame as a reference.

Subsequently, the specifying function 161 eliminates the harmonic component based on the fixed position. In this situation, the harmonic component based on the fixed position denotes, for example, a harmonic component derived from a tissue (a fixed tissue) of the patient P or a harmonic component derived from bubbles stagnating inside the body (stagnant bubbles). For example, in a liver tissue, it is known that bubbles may be taken into Kupffer cells, get fixated, and become stagnant bubbles. For this reason, the specifying function 161 eliminates the harmonic component based on the fixed position from the contrast enhanced image data.

For example, with respect to the contrast enhanced imaged data in which the tissue movements have been corrected, the specifying function 161 eliminates the harmonic component based on the fixed position, on the basis of a statistical process performed on signals in the frame direction. In one example, the specifying function 161 calculates a variance of pixel values (signal values) in the pieces of contrast enhanced image data in the frames from the n-th frame to the (n−10)-th frame. In this situation, when the calculated variance value is large, it means that the signal value of the pixel changes over the course of time. Accordingly, it is determined that the harmonic component of the pixel is based on a moving member (i.e., a bubble). On the contrary, when the calculated variance value is small, it means that the signal value of the pixel does not change over the course of time. Accordingly, it is determined that the harmonic component of the pixel is based on a fixed position. For this reason, the specifying function 161 compares the calculated variance value with a threshold value and further eliminates the harmonic component of such a pixel of which the calculated variance value is smaller than the threshold value, as a harmonic component based on the fixed position.

In this manner, the specifying function 161 eliminates the harmonic component based on the fixed position, from the contrast enhanced image data in which the movements of the tissue have been corrected. In the explanation above, the example is explained in which the variance value is calculated by using the signal values in the frames from the n-th frame to the (n−10)-th frame; however, possible embodiments are not limited to this example. For instance, the specifying function 161 may calculate a variance value by using signal values corresponding to an arbitrary number of frames. Further, for example, the specifying function 161 may calculate a variance value by using signal values in two arbitrary frames. For example, the specifying function 161 may calculate a variance value by using signal values in the two frames that are the n-th frame and the (n−10)-th frame. When a variance value is calculated by using two frames, it is preferable to use pieces of data in two frames that are apart from each other by a number of frames, rather than two consecutive frames.

Further, in the explanation above, the example is explained in which the variance value of the signal values in the plurality of frames is calculated and compared, as a statistical process performed on the signals in the frame direction; however, possible embodiments are not limited to this example. For instance, in place of the variance value, the specifying function 161 may calculate a statistical value expressing dispersion such as a standard deviation or a standard error, so as to be compared with a threshold value.

Further, the specifying function 161 specifies the positions of the bubbles. For example, the specifying function 161 specifies the positions of the bubbles (bubble positions) by generating contrast enhanced image data from which the harmonic component based on the fixed position is eliminated.

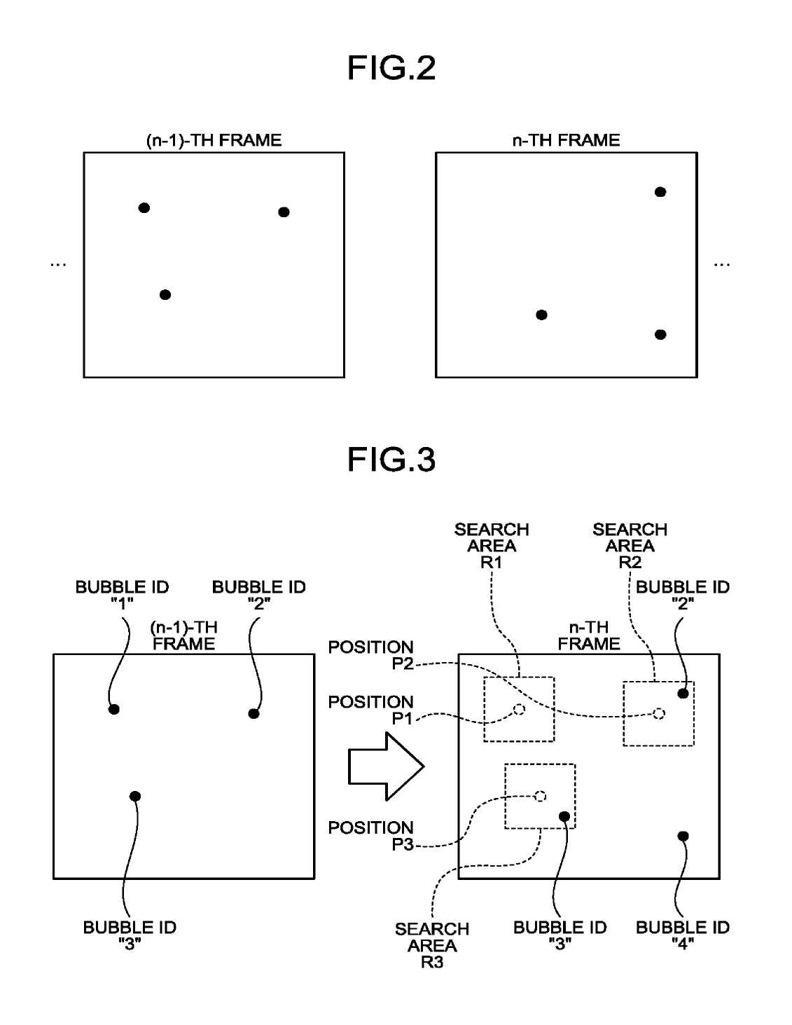

FIG. 2 is a drawing for explaining a process performed by the specifying function 161 according to the first embodiment. FIG. 2 illustrates contrast enhanced image data in which the movements of the tissue have been corrected and from which the harmonic component based on the fixed position has been eliminated. In FIG. 2, the black dots indicate bubble positions.

As illustrated in FIG. 2, every time a piece of contrast enhanced image data is generated, the specifying function 161 generates a piece of contrast enhanced image data in which the movements of the tissue have been corrected and from which the harmonic component based on the fixed position have been eliminated. For example, when a piece of contrast enhanced image data in the n-th frame is generated, the specifying function 161 generates the piece of contrast enhanced image data illustrated in FIG. 2, by correcting the movements of the tissue and eliminating a harmonic component based on the fixed position, from the piece of contrast enhanced image data in the n-th frame. After that, the specifying function 161 specifies, in the generated piece of contrast enhanced image data, the positions (coordinates) of such pixels that each have a brightness level equal to or higher than a threshold value, as bubble positions. In the example illustrated in FIG. 2, the specifying function 161 specifies the positions indicated with the black dots as the bubble positions. In this situation, it is also acceptable to perform the threshold value judging process on the contrast enhanced image data by using pixel values or signal intensities obtained by performing a filtering process that emphasizes the positions of the bubbles.

In the manner described above, the specifying function 161 specifies the bubble positions. In other words, the specifying function 161 is configured to specify the bubble positions in each of the images in the time series. In the explanation above, the example using the contrast enhanced image data generated by the specifying function 161 is explained; however, the present disclosure is not limited to displaying such contrast enhanced image data on the display device 103. In other words, it is also possible to execute the process of the specifying function 161 as an internal process of the processing circuitry 160 without having the contrast enhanced image data displayed on the display device 103.

The setting function 162 is configured to set a search area in a second medical image by referring to the positions of the contrast agent bubbles in a first medical image. For example, on the basis of the bubble positions in a previous frame, the setting function 162 sets a search area in a current frame. The setting function 162 is an example of a setting unit.

FIG. 3 is a drawing for explaining a process performed by the setting function 162 according to the first embodiment. In each of the pieces of contrast enhanced image data in the (n−1)-th frame and the n-th frame illustrated in FIG. 3, three bubbles are rendered. To the bubbles rendered in the piece of contrast enhanced image data in the (n−1)-th frame, bubble IDs “1”, “2”, and “3” are appended. Each of the bubble IDs is an identification number used for identifying the bubble.

As illustrated in FIG. 3, in the piece of contrast enhanced image data in the n-th frame, the setting function 162 identifies the positions corresponding to the bubble positions in the (n−1)-th frame. After that, the setting function 162 sets an area having a predetermined size and a predetermined shape and being centered on each of the specified positions as a search area.

More specifically, the setting function 162 obtains the coordinates of the bubble identified with the bubble ID “1” in the (n−1)-th frame. Subsequently, in the piece of contrast enhanced image data in the n-th frame, the setting function 162 specifies a position corresponding to the obtained coordinates of the bubble identified with the bubble ID “1”, as a position P1. After that, the setting function 162 sets a rectangular area having the predetermined size and being centered on the position P1, as a search area R1. Further, the setting function 162 obtains the coordinates of the bubble identified with the bubble ID “2” in the (n−1)-th frame. Subsequently, in the piece of contrast enhanced image data in the n-th frame, the setting function 162 specifies a position corresponding to the obtained coordinates of the bubble identified with the bubble ID “2”, as a position P2. After that, the setting function 162 sets a rectangular area having the predetermined size and being centered on the position P2, as a search area R2. Further, the setting function 162 obtains the coordinates of the bubble identified with the bubble ID “3” in the (n−1)-th frame. Subsequently, in the piece of contrast enhance image data in the n-th frame, the setting function 162 specifies a position corresponding to the obtained coordinates of the bubble identified with the bubble ID “3”, as a position P3. After that, the setting function 162 sets a rectangular area having the predetermined size and being centered on the position P3, as a search area R3.

In this manner, the setting function 162 sets the search areas in the piece of contrast enhanced image data in the n-th frame, on the basis of the bubble positions in the (n−1)-th frame. The explanation above is merely an example, and the present disclosure is not limited to this example. For instance, the position of the center of each of the search areas does not necessarily have to coincide with the bubble position in the (n−1)-th frame. Further, for example, the size and the shape of the search areas may arbitrarily be set. Further, although in the explanation above, the example is explained in which the search areas are set in the contrast enhanced image data, the present disclosure is not limited to displaying the contrast enhanced image data on the display device 103. In other words, it is also possible to execute the process of the setting function 162 as an internal process of the processing circuitry 160 without having the contrast enhanced image data displayed on the display device 103.

The first calculating function 163 is configured to calculate movement information of moving members, on the basis of the positions of the moving members. For example, on the basis of the positions of the contrast agent bubbles in the first medical image and in the second medical image, the first calculating function 163 calculates vectors expressing moving of the contrast agent bubbles. The first calculating function 163 calculates the vectors on the basis of the positions of the contrast agent bubbles in the search areas and the positions of the contrast agent bubbles referenced for setting the search areas. In this situation, the first calculating function 163 is an example of a computing unit. Further, the first calculating function 163 serving as an obtaining unit is configured to obtain the movement information of the moving members on the basis of the positions of the moving members.

First, the first calculating function 163 performs a tracking process on the bubbles. The tracking process is a process for determining whether each of the bubbles has moved, disappeared, or newly appeared, by conjecturing a corresponding relationship between the bubble position in the (n−1)-th frame and the bubble position in the n-th frame.

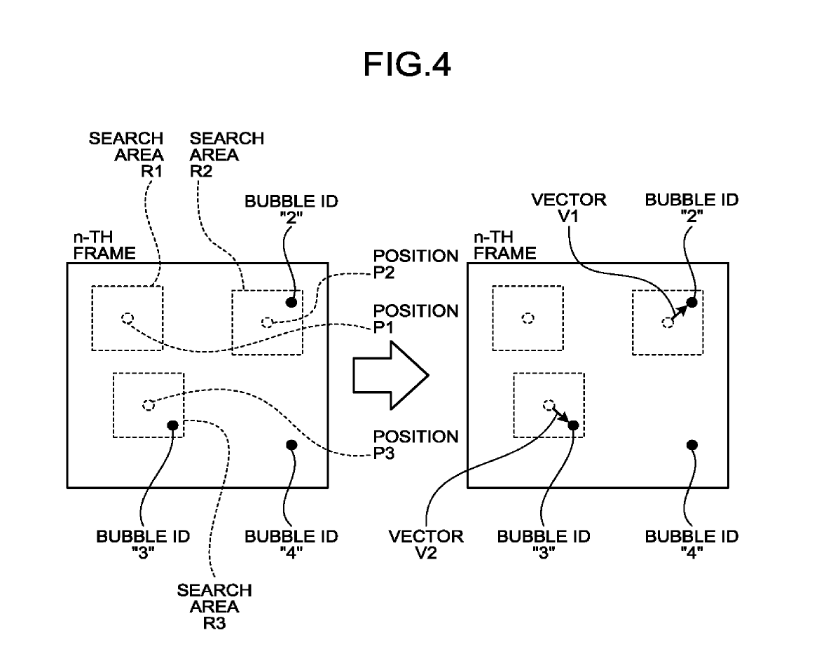

FIG. 4 is a drawing for explaining a process performed by the first calculating function 163 according to the first embodiment. On the left-hand side of FIG. 4 is a piece of contrast enhanced image data in the n-th frame in which the search areas R1 to R3 were set by the setting function 162.

As illustrated in the left section of FIG. 4, there is no bubble in the search area R1. In this situation, the search area R1 is an area that was set to be centered on the position P1 corresponding to the position of the bubble identified with the bubble ID “1” in the (n−1)-th frame. In that situation, the first calculating function 163 determines that a bubble corresponding to the bubble identified with the bubble ID “1” in the (n−1)-th frame is not present in the n-th frame. In other words, the first calculating function 163 determines that the bubble identified with the bubble ID “1” in the (n−1)-th frame disappeared in the n-th frame. As a result, the first calculating function 163 causes the bubble identified with the bubble ID “1” in the (n−1)-th frame to disappear.

Further, there is one bubble in the search area R2. In this situation, the search area R2 is an area that was set to be centered on the position P2 corresponding to the position of the bubble identified with the bubble ID “2” in the (n−1)-th frame. In that situation, the first calculating function 163 determines that the bubble in the search area R2 is a bubble corresponding to the bubble identified with the bubble ID “2” in the (n−1)-th frame. In other words, the first calculating function 163 determines that the bubble in the search area R2 is the bubble that moved from the position P2. As a result, the first calculating function 163 assigns the bubble ID “2” in the (n−1)-th frame to the bubble in the search area R2 (see the right section of FIG. 4).

Further, there is one bubble in the search area R3. In this situation, the search area R3 is an area that was set to be centered on the position P3 corresponding to the position of the bubble identified with the bubble ID “3” in the (n−1)-th frame. In that situation, the first calculating function 163 determines that the bubble in the search area R3 is a bubble corresponding to the bubble identified with the bubble ID “3” in the (n−1)-th frame. In other words, the first calculating function 163 determines that the bubble in the search area R3 is the bubble that moved from the position P3. As a result, the first calculating function 163 assigns the bubble ID “3” in the (n−1)-th frame to the bubble in the search area R3 (see the right section of FIG. 4).

Further, when there is a bubble that is not included in any of the search areas R1 to R3, the first calculating function 163 determines that the bubble is a bubble that newly appeared in the n-th frame. In the example illustrated in FIG. 4, the bubble at the bottom right in the n-th frame is a bubble that is not included in any of the search areas. In that situation, the first calculating function 163 determines that the bubble at the bottom right in the n-th frame is a bubble that newly appeared. As a result, the first calculating function 163 issues a new bubble ID “4” and assigns the bubble ID “4” to the bubble that newly appeared.

There may be some situations where there are two or more bubbles in a search area. In that situation, the first calculating function 163 may determine either a bubble positioned closest to the bubble position in the (n−1)-th frame referenced for setting the search area or a bubble that has the most similar shape, as the bubble that moved from the (n−1)-th frame (i.e., the bubble after the move). Alternatively, the first calculating function 163 may determine a bubble having the highest score based on the distance and the shape thereof, as the bubble that moved from the (n−1)-th frame.

Further, even when there is only one bubble in a search area, it is also acceptable to perform the process of comparing the shapes of the bubbles between the (n−1)-th frame and the n-th frame. In that situation, when the degree of similarity is low (lower than a predetermined threshold value), the two bubbles are identified as two separate bubbles. In that situation, the first calculating function 163 determines that the bubble in the (n−1)-th frame disappeared, whereas the bubble in the n-th frame newly appeared.

Subsequently, the first calculating function 163 calculates the vectors expressing the moving of the contrast agent bubbles, on the basis of the positions of the contrast agent bubbles in the current frame and the positions of the contrast agent bubbles in a previous frame. For example, the first calculating function 163 calculates the vectors with respect to such bubbles to each of which a bubble ID was assigned in succession in the (n−1)-th frame as well as in the n-th frame.

In the example illustrated in FIG. 4, the bubbles identified with the bubble IDs “2” and “3” are bubbles to each of which a bubble ID was assigned in succession in the (n−1)-th frame as well as in the n-th frame. In that situation, the first calculating function 163 calculates a vector V1 starting at the position P2 (a starting point) and ending at the position of the bubble identified with the bubble ID “2” (an ending point) in the n-th frame, in the right section of FIG. 4. In this situation, the vector V1 indicates the direction in which the bubble moved and the moving velocity with which the bubble moved. In this situation, the moving velocity of the bubble is calculated by converting the distance between the starting point and the ending point into a length in real space (i.e., a pitch size) and dividing the length by the frame interval. Similarly, with respect to the bubble identified with the bubble ID “3”, the first calculating function 163 calculates a vector V2 starting at the position P3 (a starting point) and ending at the position of the bubble identified with the bubble ID “3” (an ending point) in the n-th frame. In other words, the first calculating function 163 is configured to calculate the moving velocity of the contrast agent from a difference in the temporal phase between a first temporal phase and a second temporal phase and the length of a vector in real space.

In this manner, the first calculating function 163 calculates the vectors expressing the moving of the bubbles. In other words, the first calculating function 163 serving as an obtaining unit is configured to calculate the vectors expressing the moving of the bubbles, by tracking the positions of the bubbles in each of the images in the time series. The explanation above is merely an example, and possible processes that can be performed by the first calculating function 163 are not limited to those in this example. For instance, in the explanation above, the example is explained in which each of the vectors is calculated by using the displacement (the distance) of the positions of the bubble between the two frames that are next to each other; however possible embodiments are not limited to this example. For instance, the first calculating function 163 may calculate each of the vectors by using a displacement of a bubble between two arbitrary temporal phases. In relation to this, as for the process of calculating a vector expressing the moving of a bubble, it is possible to adopt any of the processes disclosed in Japanese Patent Application Laid-open No. 2018-015155.

Further, in the explanation above, the example is explained in which the vectors are calculated as the movement information of the moving members; however, possible embodiments are not limited to this example. For instance, as the movement information of each of the moving members, the first calculating function 163 is capable of calculating at least one selected from among: the velocity, a displacement, the moving direction, and a time period before arrival. In this situation, the displacement denotes a moving amount (the distance) of the moving member represented by a bubble, between two arbitrary temporal phases. The velocity denotes a displacement per arbitrary unit time period (e.g., one frame, one second, or the like). The moving direction denotes an angle with respect to an arbitrary direction (e.g., the vertically upward direction in the image) used as a reference. Further, the time period before arrival denotes a time period during which the bubble is detected that is expressed by using an arbitrary temporal phase as a reference. For example, the time period before arrival denotes a time period from a point in time at which the imaging process was started, to a point in time at which each of the bubbles was detected. Alternatively, when the time at which each of the bubbles was detected for the first time is used as a reference temporal phase, the time period before arrival may denote, for example, an elapsed time period since the reference temporal phase. In other words, the movement information includes a component in a direction different from the direction of the ultrasound scan performed on the scan region. For example, the movement information includes the moving directions of the individual bubbles.

For example, in the explanation above, the example is explained in which the vectors are calculated in the contrast enhanced image data; however, the present disclosure is not limited to displaying the contrast enhanced image data on the display device 103. In other words, it is also possible to execute the process of the first calculating function 163 as an internal process of the processing circuitry 160 without having the contrast enhanced image data displayed on the display device 103.

The second calculating function 164 is configured to calculate a moment of second or higher order related to the movement information of the moving members, with respect to at least a part of the scan region. For example, with respect to a point (a position) designated by the operator, the second calculating function 164 calculates a variance value of the velocity values of the bubble in the frame direction (the time direction). The variance value of the velocity values of the bubble in the time direction is an example of the moment of second or higher order (a moment in two or more dimensions) related to the movement information of the moving member. The second calculating function 164 is an example of the obtaining unit configured to obtain a moment of second or higher order.

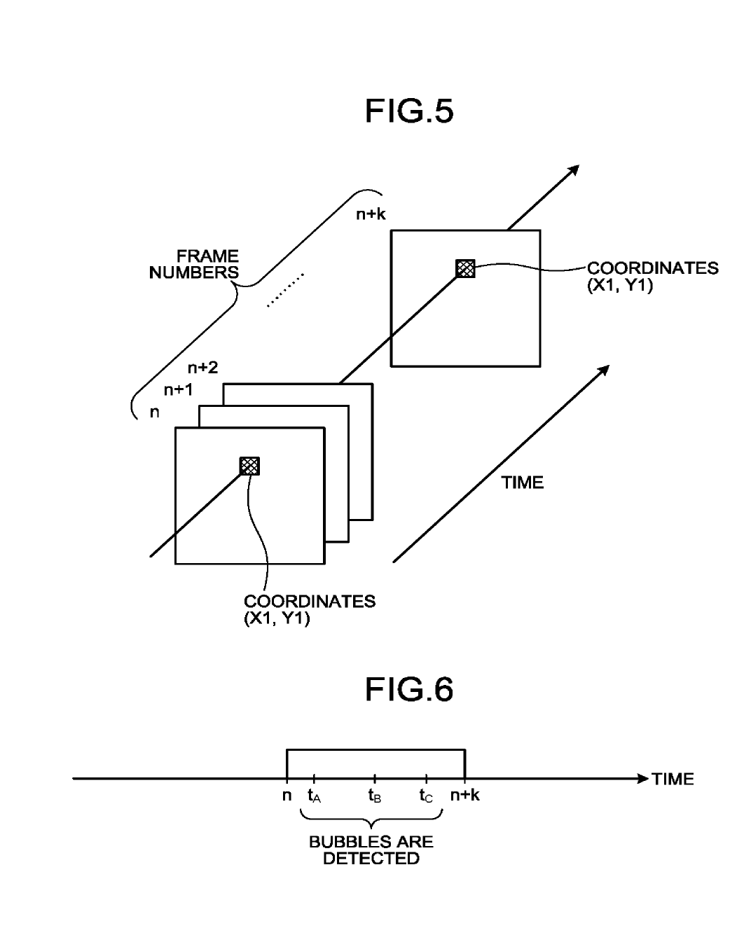

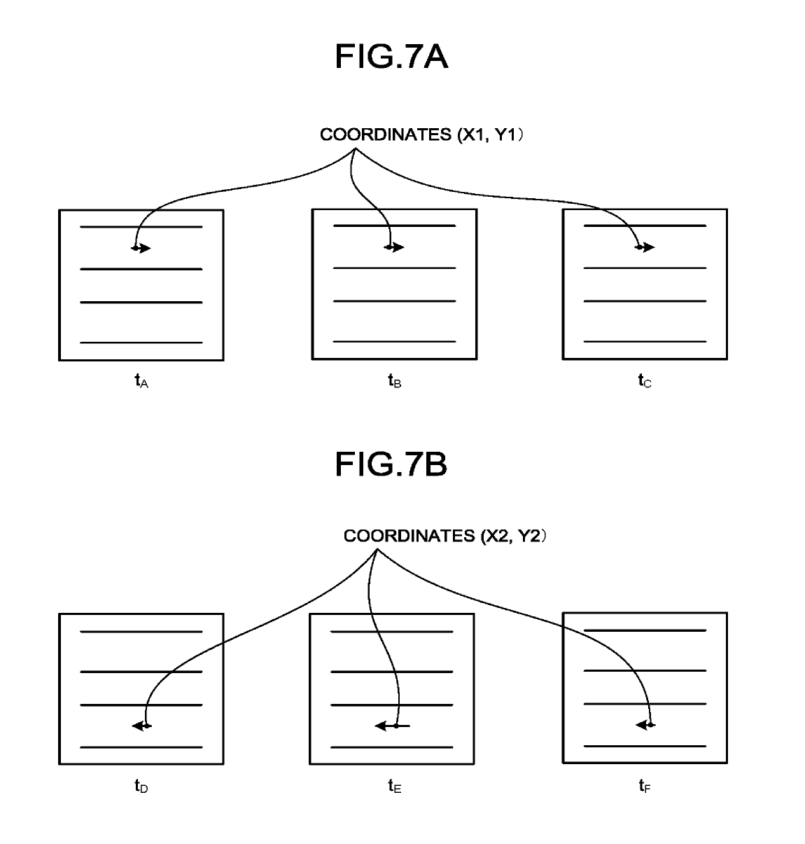

FIGS. 5, 6, 7A, and 7B are drawings for explaining a process performed by the second calculating function 164 according to the first embodiment. FIG. 5 illustrates images in a time series from the n-th frame to an (n+k)-th frame. FIG. 6 illustrates a process of detecting frames in which bubbles are present. FIG. 7A illustrates a vector of a bubble detected at coordinates (X1,Y1) in a vein. FIG. 7B illustrates a vector of a bubble detected at coordinates (X2,Y2) included in an artery. In this situation, the bubble detected at the coordinates (X1,Y1) is moving in the direction toward the right-hand side of FIG. 7A. In contrast, the bubble detected at the coordinates (X2,Y2) is moving in the direction toward the left-hand side of FIG. 7B.

As illustrated in FIG. 5, for example, with respect to the coordinates (X1,Y1), the second calculating function 164 calculates a variance value of the velocity values of the bubble in the time period from the n-th frame to the (n+k)-th frame. In this situation, a bubble may not necessarily be present at the coordinates (X1,Y1) in all the frames from the n-th frame to the (n+k)-th frame. For this reason, the second calculating function 164 detects the frames in which a bubble is present at the coordinates (X1,Y1) during the time period from the n-th frame to the (n+k)-th frame. In this situation, k denotes a natural number.

As illustrated in FIG. 6, for example, the second calculating function 164 detects the frames in which a bubble is present at the coordinates (X1,Y1), while using, as a processing target, images of which the quantity is equal to (k+1) that are included in the time period from the n-th frame to the (n+k)-th frame, from among the pieces of contrast enhanced image data in which the positions of the bubbles were specified by the specifying function 161. In the example illustrated in FIG. 6, the second calculating function detects three frames such as t_{A}, t_{B}, and t_{C}, as the frames in each of which a bubble is present at the coordinates (X1,Y1). In FIG. 6, the horizontal axis corresponds to the time direction. Further, t_{A}, t_{B}, and t_{C} denote numbers that satisfy “n<t_{A}<t_{B}<t_{C}<n+k”.

As illustrated in FIG. 7A, a bubble is detected at the coordinates (X1,Y1) in the images in the three frames identified as t_{A}, t_{B}, and t_{C}. In FIG. 7A, each of the arrows represents a vector of the bubble at the center position of the arrow. More specifically, the direction of each of the arrows corresponds to the direction of the vector, whereas the length of each of the arrows corresponds to the displacement (the moving amount) of the vector.

For example, the second calculating function 164 calculates a variance value by using Expression (1) presented below while using, as a processing target, the three pieces of movement information of the bubble detected in the three frames identified as t_{A}, t_{B}, and t_{C}. In Expression (1), σ² denotes the variance value, whereas V(t) denotes the velocity of the bubble. Further, the letter t denotes time, whereas the letter μ denotes an average velocity value. In the example in FIG. 7A, the letter μ denotes an average value of the three velocity values of the bubble detected in the three frames identified as t_{A}, t_{B}, and t_{C}. N denotes the number of samples. In the example in FIG. 7A, N is equal to 3. The letter “s” denotes the time of the starting point, whereas the letter “e” denotes the time of the ending point.

$${σ}^{2} = \frac{1}{N} {\int }_{t = s}^{e} {( \vert v ( t ) \vert - μ )}^{2} dt = \frac{1}{N} {\sum }_{t = s}^{e} {( \vert v ( t ) \vert - μ )}^{2} dt \qquad ( 1 )$$

In this situation, it is known that, in veins, bubbles move with substantially constant velocity, because veins are not easily impacted by the pulsation. For this reason, in the example illustrated FIG. 7A, the bubble detected in the three frames identified as t_{A}, t_{B}, and t_{C} are moving with mutually the same levels of velocity approximately.

In contrast, it is known that, in arteries, the velocity of the blood flow (i.e., bubbles) varies, because arteries are impacted by the pulsation. For this reason, as illustrated in FIG. 7B, the bubble detected at the coordinates (X2,Y2) included in the artery moves with mutually-different levels of velocity. More specifically, the bubble detected in the frame identified as t_{E} is moving with higher velocity than the bubble detected in each of the two frames identified as t_{D} and t_{F}. In this situation, because the process of detecting the three frames identified as t_{D}, t_{E}, and t_{F} is the same as the process explained with reference to FIGS. 5 and 6, the explanation thereof will be omitted.

In other words, the second calculating function 164 calculates a smaller variance value for each of the bubbles detected in veins, compared to the variance value calculated for each of the bubble detected in arteries. That is to say, the second calculating function 164 calculates a larger variance value for each of the bubbles detected in arteries, compared to the variance value calculated for each of the bubbles detected in veins.

In this manner, the second calculating function 164 is configured to calculate the variance value of the velocity values of the bubble in the frame direction (the time direction) with respect to the point (the position) designated by the operator. The explanation above is merely an example, and the process performed by the second calculating function 164 is not limited to this example. For instance, possible mathematical formulae that can be used by the second calculating function 164 are not limited to Expression (1) presented above. Other mathematical formulae that can be used by the second calculating function 164 will be explained later in modification examples.

Further, in the explanation above, the example is explained in which the variance value of the velocity values of the bubble is calculated with respect to the point (the position) designated by the operator; however, possible embodiments are not limited to this example. For instance, the second calculating function 164 is also capable of calculating a moment of second or higher order, with respect to certain positions in a region of interest within the scan region. For example, the region of interest may be set within the scan region by the operator.

The display controlling function 165 is configured to output the information calculated by the second calculating function 164. For example, the display controlling function 165 causes the display device 103 to display a second image structured with pixels each having a pixel value expressing the moment of second or higher order. In that situation, the image generating circuitry 130 is configured to generate the second image structured with the pixels each having a pixel value expressing the moment of second or higher degree.

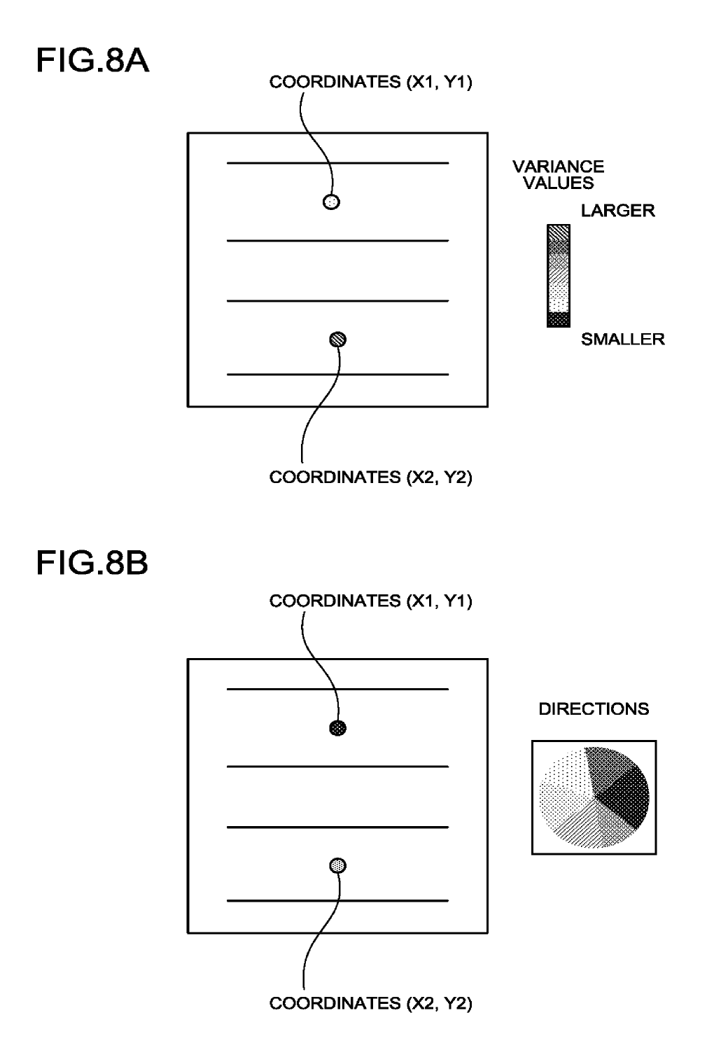

FIGS. 8A and 8B are drawings for explaining a process performed by the display controlling function 165 according to the first embodiment. FIG. 8A illustrates an image in which a pixel value corresponding to the variance value at each set of coordinates is assigned to the set of coordinates. FIG. 8B illustrates an image in which a pixel value corresponding to the direction at each set of coordinates is assigned to the set of coordinates. Each of the images illustrated in FIGS. 8A and 8B is an example of the second image.

In the example illustrated in FIG. 8A, the image generating circuitry 130 generates the image in which a pixel value corresponding to the variance value at each set of coordinates is assigned to the set of coordinates. After that, the display controlling function 165 displays the image generated by the image generating circuitry 130 together with a color scale of the variance values. The color scale of the variance values (in the right section of FIG. 8A) is a scale indicating changes in the pixel values in correspondence with changes in the variance values.

In FIG. 8A, at the coordinates (X1,Y1) included in the vein, a variance value smaller than that at the coordinates (X2,Y2) was calculated. For this reason, a pixel value corresponding to a smaller variance value is assigned to the coordinates (X1,Y1), compared to the pixel value assigned to the coordinates (X2,Y2). Conversely, at the coordinates (X2,Y2) included in the artery, a variance value larger than that at the coordinates (X1,Y1) was calculated. For this reason, a pixel value corresponding to a larger variance value is assigned to the coordinate (X2,Y2), compared to the pixel value assigned to the coordinates (X1,Y1).

In the example illustrated in FIG. 8B, the image generating circuitry 130 generates the image in which a pixel value corresponding to the direction of the vector at each set of coordinates is assigned to the set of coordinates. After that, the display controlling function 165 displays the image generated by the image generating circuitry 130 together with a color scale of the directions. The color scale of the directions (in the right section of FIG. 8B) is a scale in which a pixel value corresponding to each of the various directions in 360 degrees from the center of the circle is assigned to a corresponding one of the various positions in the circle. More specifically, in the color scale of the directions, for example, darker pixel values are assigned to the directions toward the right, whereas lighter pixel values are assigned to the directions toward the left.

In this situation, in FIG. 8B, the bubble detected at the coordinates (X1,Y1) moves in the direction toward the right in the drawing. For this reason, a darker pixel value is assigned to the coordinates (X1,Y1) compared to the pixel value assigned to the coordinates (X2,Y2). In contrast, the bubble detected at the coordinates (X2,Y2) moves in the direction toward the left in the drawing. For this reason, a lighter pixel value is assigned to the coordinates (X2,Y2) compared to the pixel value assigned to the coordinates (X1,Y1).

In the manner described above, the display controlling function 165 causes the display device 103 to display the image in which a pixel value corresponding to either the variance value or the direction is assigned to each set of coordinates. In this situation, besides the pixel values corresponding to the variance values or the directions, the display controlling function 165 is also capable of displaying other types of images in which a pixel value corresponding to any other parameter calculated by the second calculating function 164 is assigned.

Further, in the explanation above, the example is explained in which the pixel value corresponding to the variance value (or the direction) is assigned to the point (the position) designated by the operator; however, possible embodiments are not limited to this example. For instance, the display controlling function 165 is also capable of displaying an image (a parametric image) in which, to each of various positions in a region of interest within the scan region, a pixel value corresponding to a parameter in the position is assigned.

Further, in the explanation above, the example is explained in which the calculation result obtained by the second calculating function 164 is output as the image; however, possible embodiments are not limited to this example. For instance, the display controlling function 165 may output the calculation result obtained by the second calculating function 164 as numerical values (text data). Further, the output destination to which the display controlling function 165 outputs the information does not necessarily have to be the display device 103 and may be a storage medium or another information processing apparatus, for example.

Further, possible embodiments of the color scales are not limited to those illustrated in FIGS. 8A and 8B. For example, the display controlling function 165 may display the information by using a circular color scale expressing the variance values and the directions. The circular color scale in this example is a scale to which colors corresponding to the directions of the vectors and the darkness/lightness levels corresponding to the variance values are assigned. In other words, for the directions of the vectors, a color (a hue) corresponding to each of the various directions in 360 degrees from the center of the circle is assigned to a corresponding one of the various positions in the circle. As for the variance values, a darkness/lightness level corresponding to the magnitude of each of the variance values is assigned to a corresponding one of the various positions in the circle, in such a manner that the closer the position is to the center of the circle, the darker is the color, and conversely, the closer the position is to the circumference of the circle, the lighter is the color.

Alternatively, for example, the display controlling function 165 may display trajectories of the tracked bubbles by using lines and may assign a pixel value corresponding to the variance value to each of the points on the lines.

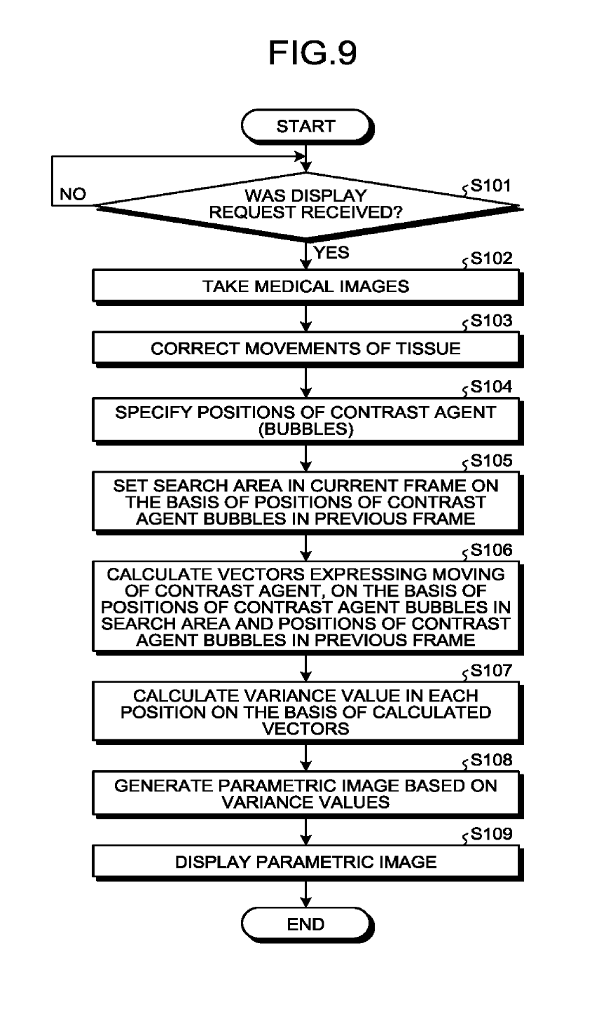

FIG. 9 is a flowchart for explaining a processing procedure performed by the ultrasound diagnosis apparatus 1 according to the first embodiment. The processing procedure illustrated in FIG. 9 is started, for example, when a display request is received from the operator.

As illustrated in FIG. 9, for example, when the input device 102 receives a display request from the operator (step S101: Yes), the processing circuitry 160 starts the processes at step S102 and thereafter. Until the display request is received (step S101: No), the processing circuitry 160 does not start the processes described below and is in a standby state.

When a display request is received, the transmission and reception circuitry 110 takes medical images (step S102). For example, the transmission and reception circuitry 110 causes the ultrasound probe 101 to perform an ultrasound scan for taking ultrasound image data, under control of the processing circuitry 160. Further, the signal processing circuitry 120 and the image generating circuitry 130 take, in a real-time manner, contrast enhanced image data and tissue image data, by using the reflected-wave data acquired by the transmission and reception circuitry 110.

Subsequently, the specifying function 161 corrects movements of the tissue (step S103). For example, the specifying function 161 calculates a correction amount used for arranging the coordinate system of the piece of tissue image data in the (n+k)-th frame to coincide with the coordinate system of the piece of tissue image data in the (n+k−1)-th frame. After that, the specifying function 161 corrects the coordinate system of the piece of contrast enhanced image data in the (n+k)-th frame by using the calculated correction amount. Further, the specifying function 161 eliminates the harmonic component based on the fixed position. For example, from the contrast enhanced image data in which the movements of the tissue have been corrected, the specifying function 161 eliminates the harmonic component based on the fix position, on the basis of a statistical process performed on the signals in the frame direction.

Subsequently, the specifying function 161 specifies the positions of the contrast agent (bubbles) (step S104). For example, the specifying function 161 specifies the bubble positions by generating contrast enhanced image data from which the harmonic component based on the fixed position is eliminated.

After that, the setting function 162 sets search areas in the current frame, on the basis of the positions of the contrast agent bubbles in a previous frame (step S105). For example, the setting function 162 sets the search areas in the piece of contrast enhanced image data in the (n+k)-th frame, on the basis of the bubble positions in the (n+k−1)-th frame.

After that, the first calculating function 163 calculates vectors expressing the moving of the contrast agent bubbles, on the basis of the positions of the contrast agent bubbles in the search areas and the positions of the contrast agent bubbles in the previous frame (step S106). For example, the first calculating function 163 calculates a vector of each of the bubbles to which a bubble ID is assigned in succession in the (n+k−1)-th frame as well as in the (n+k)-th frame.

After that, the second calculating function 164 calculates a variance value in each of the positions, on the basis of the calculated vectors (step S107). For example, with respect to each of the points (or an area) designated by the operator, the second calculating function 164 calculates a variance value of the velocity values of the bubble in the frame direction from the n-th frame to the (n+k)-th frame.

Subsequently, the image generating circuitry 130 generates a parametric image based on the variance values (step S108). For example, the image generating circuitry 130 generates the parametric image by assigning a pixel value corresponding to the variance value in each of the positions calculated by the second calculating function 164, to the position.

After that, the display controlling function 165 displays the parametric image (step S109). For example, the display controlling function 165 causes the display device 103 to display the parametric image generated by the image generating circuitry 130. Subsequently, the processing circuitry 160 ends the processing procedure illustrated in FIG. 9.

The explanation above is merely an example, and possible embodiments are not limited to this example. For instance, the process at step S103 does not necessarily have to be performed. Further, when the process is performed in a retroactive manner by using ultrasound image data that has already been taken, the process of taking the medical images at step S102 shall not be performed.

As explained above, in the ultrasound diagnosis apparatus 1 according to the first embodiment, the image generating circuitry 130 is configured to generate the images in the time series on the basis of the result of the scan performed on the scan region. Further, the specifying function 161 is configured to specify the positions of the moving members included in the scan region, with respect to each of the images in the time series. Subsequently, on the basis of the positions of the moving members, the first calculating function 163 is configured to calculate the movement information of the moving members. With respect to at least a part of the scan region, the second calculating function 164 is configured to calculate the moment of second or higher order related to the movement information of the moving members. With these arrangements, the ultrasound diagnosis apparatus 1 is able to accurately evaluate the dynamics of the blood flows.

For example, the ultrasound diagnosis apparatus 1 according to the first embodiment is configured to track each of the bubbles used as the contrast agent, unlike conventional contrast enhanced echo methods and Micro Flow Imaging (MFI) methods by which a blood vessel is rendered as a whole. Accordingly, the ultrasound diagnosis apparatus 1 is able to quantitatively display the directions and the moving velocity values in which and with which the bubbles of the contrast agent flow, by using the vectors.

Further, for example, the ultrasound diagnosis apparatus 1 makes it possible to easily distinguish arteries and veins from each other, by calculating the variance value of the velocity values of each of the bubbles in the time direction. This technique is based on the notion that the blood flow in an artery exhibits changes in the velocity in the time direction due to impacts of the pulsation, whereas the blood flow in a vein is not easily impacted by the pulsation and thus flows with substantially constant velocity. By browsing the parametric image based on the variance values of the moving velocity values of the bubbles, the operator is able to easily distinguish arteries and veins from each other in the scan region.

As explained above, the ultrasound diagnosis apparatus 1 is able to evaluate the dynamics of the blood flows in a stable manner, regardless of the hospital or the medical doctor using the apparatus. In particular, the ultrasound diagnosis apparatus 1 makes it possible to quantitatively make an observation on the same patient or on the same site of the body over the course of time.

In the first embodiment, the example is explained in which the second calculating function 164 calculates the variance value of the velocity values of each of the bubbles in the time direction by using Expression (1); however, possible embodiments are not limited to this example. Accordingly, modification examples of the process performed by the second calculating function 164 will be explained below.

### First Modification Example of First Embodiment

For example, with reference to Expression (1), the example was explained in which the second calculating function 164 calculates the variance value in the time direction by using the velocity V(t) of each of the bubbles; however, possible embodiments are not limited to this example. For instance, it is also acceptable to express Expression (1) by using vectors, as presented in Expression (2) below.

$${σ}^{2} = \frac{1}{N} {\int }_{t = s}^{e} {( \vert \vec{v} ( t ) \vert - μ )}^{2} dt = \frac{1}{N} {\sum }_{t = s}^{e} {( \vert \vec{v} ( t ) \vert - μ )}^{2} dt \qquad ( 2 )$$

In Expression (2), V(t) (where V has an arrow) corresponds to a vector expressing the moving of each bubble. By using Expression (2), the second calculating function 164 is able to calculate a variance value of the vectors of each of the bubbles in the time direction.

### Second Modification Example of First Embodiment

Further, for example, with reference to Expression (1) presented above, the example was explained in which the variance value represented by the two-dimensional moment is calculated; however, possible embodiments are not limited to this example. For instance, as presented in Expression (3) below, the second calculating function 164 is also capable of calculating an n-th order moment.

$${σ}^{n} = \frac{1}{N} {\int }_{t = s}^{e} {( \vert v ( t ) \vert - μ )}^{n} dt = \frac{1}{N} {\sum }_{t = s}^{e} {( \vert v ( t ) \vert - μ )}^{n} dt \qquad ( 3 )$$

In Expression (3), n denotes the degree of dimension (which is different from “n” used above to indicate the frame number). For example, when the “n” in Expression (3) is “3 (i.e., three-dimensional)”, the second calculating function 164 calculates a level of skewness. In contrast, when the “n” in Expression (3) is “4 (i.e., four-dimensional)”, the second calculating function 164 calculates a level of kurtosis.

### Third Modification Example of First Embodiment

With reference to Expressions (1) to (3) presented above, the examples were explained in which the moment of second or higher order (an n-th order moment) around the average value is calculated; however, possible embodiments are not limited to this example. For instance, as indicated in Expression (3) presented below, the second calculating function 164 is able to calculate an n-th order moment around the origin.

$${σ}^{n} = \frac{1}{N} {\int }_{t = s}^{e} {\vert v ( t ) \vert}^{n} dt = \frac{1}{N} {\sum }_{t = s}^{e} {\vert v ( t ) \vert}^{n} dt \qquad ( 4 )$$

In Expression (4), the value at the origin is “0”. Alternatively, the second calculating function 164 is also capable of calculating an n-th order moment around an arbitrary (e.g., a median value), besides the average value or the origin. In other words, the second calculating function 164 is also capable of calculating a moment of second or higher order around one selected from among an average value, a median value, and the origin, with respect to the movement information of each of the bubbles.

### Fourth Modification Example of First Embodiment

In the explanation above, the example is explained in which the moment of second or higher order (the n-th order moment) in the time direction is calculated; however, possible embodiments are not limited to this example. For instance, the second calculating function 164 may calculate a moment of second or higher order (an n-th order moment) in a space direction as indicated in Expression (5) presented below.

$${σ}^{n} = \frac{1}{N} {\int }_{x}^{e} {\int }_{y}^{e} {( \vert v ( x , y ) \vert - μ )}^{n} dxdy = \frac{1}{N} {\sum }_{x}^{e} {\sum }_{y}^{e} {( \vert v ( x , y ) \vert - μ )}^{n} dxdy \qquad ( 5 )$$

In Expression (5), x corresponds to the horizontal direction in an image space, whereas y corresponds to the vertical direction in the image space.

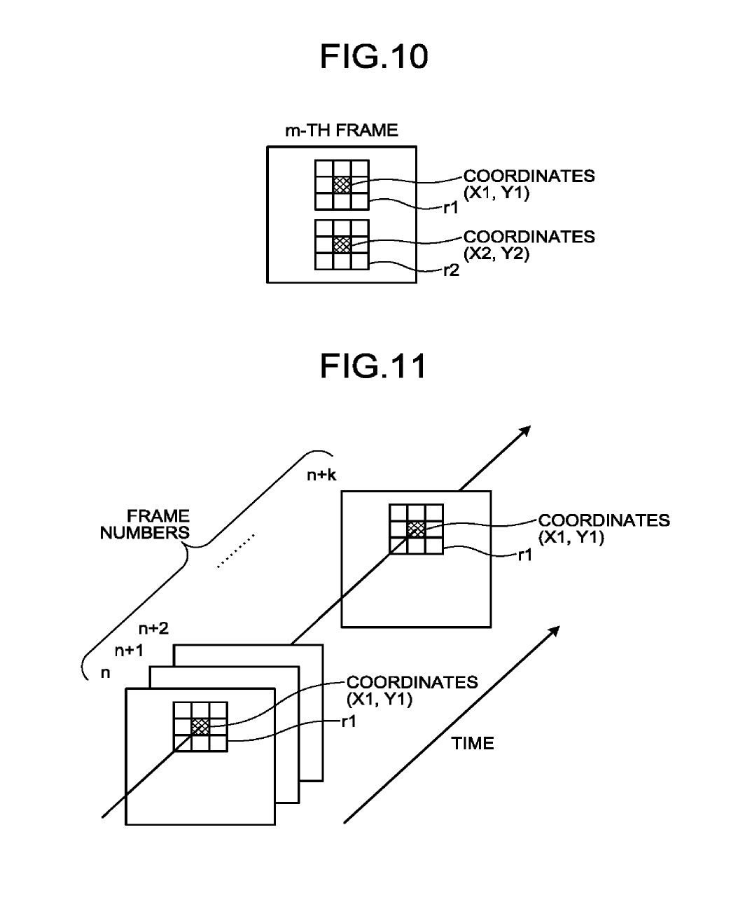

FIG. 10 is a drawing for explaining a process performed by the second calculating function 164 according to the present modification example of the first embodiment. As illustrated in FIG. 10, the second calculating function 164 calculates an n-th order moment in the space direction, by using a 3×3 region r1 centered on the coordinates (X1,Y1).

In this situation, because the blood flow in arteries has larger changes in the velocity due to impacts of the pulsation, the dispersion of velocity values among a plurality of bubbles, even at a single point in time (in a single temporal phase), is larger than that of the blood flow in veins. For this reason, the second calculating function 164 calculates a variance value in the space direction.

The illustration in FIG. 10 is merely an example, and possible embodiments are not limited to this example. For instance, also with respect to the coordinates (X2,Y2), the second calculating function 164 is capable of calculating an n-th order moment in the space direction by using a 3×3 region r2 centered on the coordinates (X2,Y2). Further, the size of each of the regions r1 and r2 does not necessarily have to be 3×3 and may be set to an arbitrary size. Further, the center of each of the regions r1 and r2 does not necessarily have to coincide with the coordinates of the processing target.

### Fifth Modification Example of First Embodiment

Further, for example, as indicated in Expression (6) presented below, the second calculating function 164 may calculate a moment of second or higher order (an n-th order moment) in the spatiotemporal direction (the time direction and the space direction).

$${σ}^{n} = \frac{1}{N} {\int }_{x}^{e} {\int }_{y}^{e} {\int }_{t}^{e} {( \vert v ( x , y , t ) \vert - μ )}^{n} dtdxdy = \frac{1}{N} {\sum }_{x}^{e} {\sum }_{y}^{e} {\sum }_{t}^{e} {( \vert v ( x , y , t ) \vert - μ )}^{n} dtdxdy \qquad ( 6 )$$

FIG. 11 is a drawing for explaining a process performed by the second calculating function 164 according to the present modification example. As illustrated in FIG. 11, the second calculating function 164 calculates an n-th order moment in the spatiotemporal direction, by using the 3×3 region r1 centered on the coordinates (X1,Y1).

For example, by using Expression (6), the second calculating function 164 calculates an n-th order moment around average values in the time direction and in the space direction. Both in the time direction and in the space direction, a larger value is calculated for a blood flow in an artery than for a blood flow in a vein. Accordingly, as for the n-th order moment in the spatiotemporal direction also, a larger value is calculated for a blood flow in an artery than for a blood flow in a vein. In other words, the second calculating function 164 calculates a variance value that is temporal, spatial, or spatiotemporal, as the moment of second or higher order.

The illustration in FIG. 11 is merely an example, and possible embodiments are not limited to this example. For instance, the size of the region r1 does not necessarily have to be 3×3 and may be set to an arbitrary size. Further, the center of the region r1 does not necessarily have to coincide with the coordinates (X1,Y1) of the processing target.

### Sixth Modification Example of First Embodiment

Further, for example, the second calculating function 164 may calculate an average value in the space direction and subsequently calculate a variance value in the time direction by using the calculated average value.

A process performed by the second calculating function 164 according to a sixth modification example of the first embodiment will be explained, with reference to FIG. 11. For example, the second calculating function 164 sets the 3×3 region r1 centered on the coordinates (X1,Y1) with respect to each of the frames from the n-th frame to the (n+k)-th frame. After that, in each of the frames, the second calculating function 164 calculates an average value of the velocity values of the bubbles included in the region r1. Subsequently, the second calculating function 164 calculates a variance value in the time direction with respect to the calculated average value, in the frame direction from the n-th frame to the (n+k)-th frame.

As explained above, the second calculating function 164 may calculate the average value in the space direction and subsequently calculate the variance value in the time direction by using the calculated average value.

### Seventh Modification Example of First Embodiment

Further, for example, the second calculating function 164 is also capable of calculating an n-th order moment around an arbitrary value, with respect to an arbitrary parameter calculated by the first calculating function 163. For example, the second calculating function 164 is capable of calculating a variance value around an average value of moving directions of the bubbles.

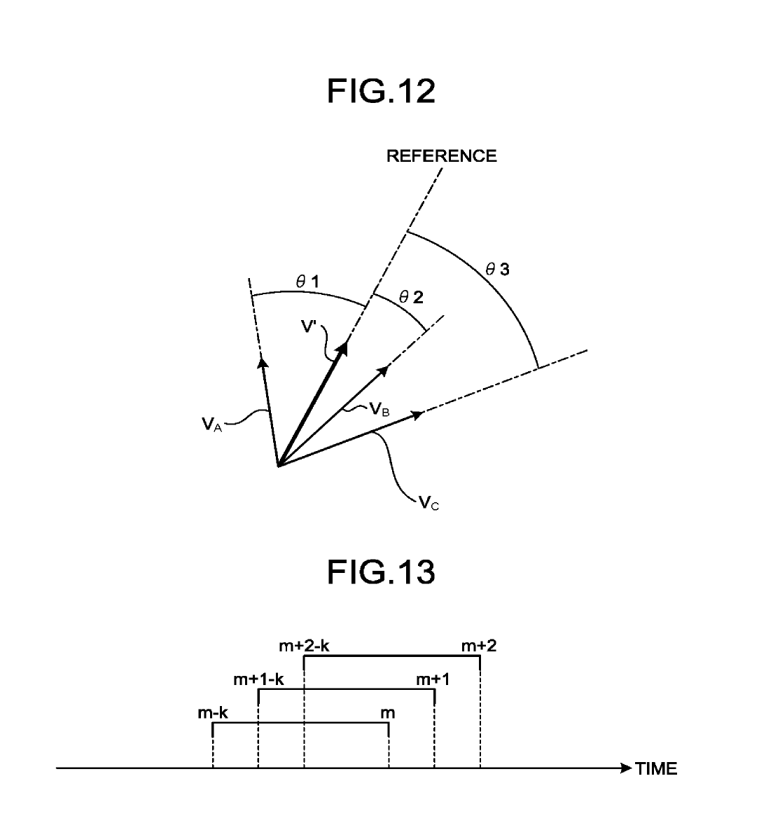

FIG. 12 is a drawing for explaining a process performed by the second calculating function 164 according to the present modification example of the first embodiment. FIG. 12 illustrates three vectors of a bubble in the time period from the n-th frame to the (n+k)-th frame, while aligning the positions of the origin with each other. In FIG. 12, for example, V_{A} denotes the vector of the bubble detected in the frame t_{A}. V_{B} denotes the vector of the bubble detected in the frame t_{B}. V_{C} denotes the vector of the bubble detected in the frame t_{C}.

As illustrated in FIG. 12, the second calculating function 164 calculates an average vector V′ of the three vectors V_{A}, V_{B}, and V_{C}. Further, the second calculating function 164 calculates a variance value of the moving directions, by using the direction indicated by the average vector V′ as a reference. More specifically, the second calculating function 164 calculates angles θ1, θ2, and θ3 formed by the average vector V′ used as a reference and the three vectors V_{A}, V_{B}, and V_{C}, respectively. After that, the second calculating function 164 calculates a variance value of the angles θ1, θ2, and θ3 as the variance value of the moving directions of the bubble.

As for moving directions of bubbles, it is considered that the lower the velocity of the blood flow is, the larger the dispersion thereof is and that the higher the velocity of the blood flow is, the closer the moving directions are to a constant value. For this reason, it is considered that the variance value of moving directions in arteries is smaller than that in veins. Conversely, it is considered that the variance value of moving directions in veins is smaller than that in arteries.

With reference to FIG. 12, the example is explained in which the variance value around the average value of the moving directions is calculated; however, possible embodiments are not limited to this example. For instance, the second calculating function 164 is also capable of calculating an n-th order moment around an arbitrary value with respect to an arbitrary parameter (velocity values, displacements, or time periods before arrival of the moving members) calculated by the first calculating function 163.

### Second Embodiment

In the first embodiment, the example is explained in which in the (n+k)-th frame, the variance value in the time direction is calculated for the frames from the n-th frame to the (n+k)-th frame; however, possible embodiments are not limited to this example. For instance, the ultrasound diagnosis apparatus 1 may sequentially perform the processes explained in the first embodiment over the course of time.

FIG. 13 is a drawing for explaining a process performed by the ultrasound diagnosis apparatus 1 according to a second embodiment. As illustrated in FIG. 13, for example, when having generated an image in an m-th frame (step S102 in FIG. 9), the ultrasound diagnosis apparatus 1 calculates a variance value of the velocity values of each of the bubbles in the time direction in the frames from the (m-k)-th frame to the m-th frame, by performing the processes at steps S103 through S109 in FIG. 9. In this situation, m and k each denote a natural number.

Subsequently, when having generated an image in the (m+1)-th frame (step S102 in FIG. 9), the ultrasound diagnosis apparatus 1 calculates a variance value of the velocity values of each of the bubbles in the time direction in the frames from the (m+1-k)-th frame to the (m+1)-th frame by performing the processes at steps S103 through S109 in FIG. 9.

Subsequently, when having generated an image in the (m+2)-th frame (step S102 in FIG. 9), the ultrasound diagnosis apparatus 1 calculates a variance value of the velocity values of each of the bubbles in the time direction in the frames from the (m+2-k)-th frame to the (m+2)-th frame, by performing the processes at steps S103 through S109 in FIG. 9.

In this manner, when the images (the pieces of contrast enhanced image data) in a time-series sequence are sequentially taken over the course of time by performing a real-time imaging process, the ultrasound diagnosis apparatus 1 repeatedly performs the processing procedure in FIG. 9, while using, as the processing target, the images in the frames from the generated frame to another frame earlier by a certain period of time. Accordingly, the ultrasound diagnosis apparatus 1 is able to display a parametric image in a real-time manner.

### Third Embodiment

Further, in the embodiments above, the example is explained in which displaying the parametric image makes it easier for the operator to distinguish the arteries and the veins from each other; however, possible embodiments are not limited to this example. For instance, the ultrasound diagnosis apparatus 1 is also capable of presenting the operator with an image in which arteries and veins are distinguished from each other by binarizing the variance values.

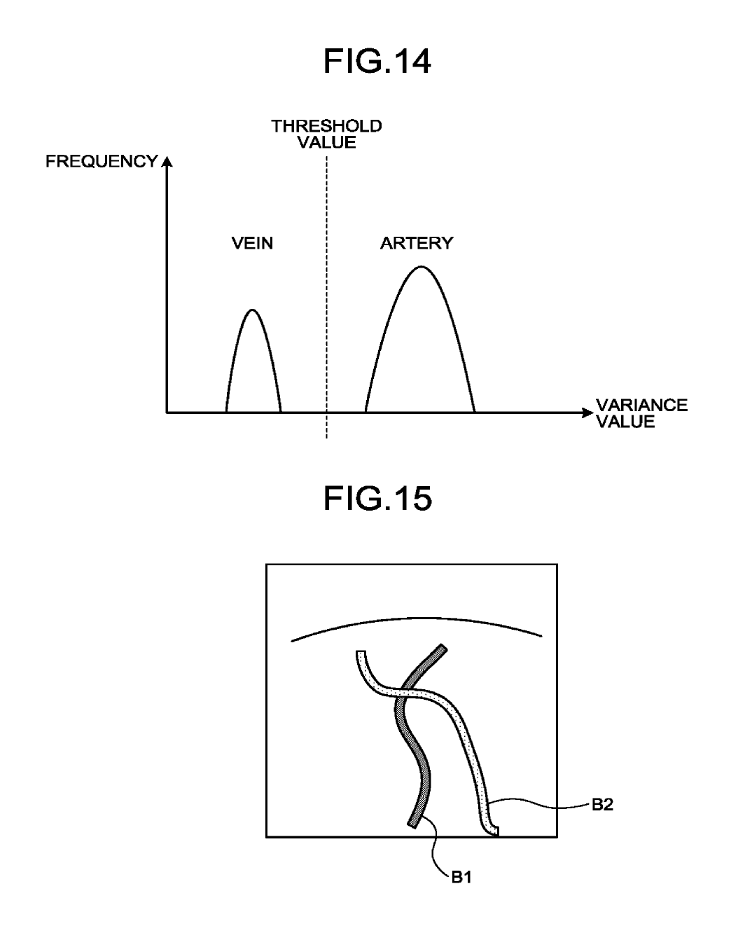

FIGS. 14, 15, and 16 are drawings for explaining a process performed by the ultrasound diagnosis apparatus 1 according to a third embodiment. As illustrated in FIG. 14, for example, the second calculating function 164 calculates a variance value with respect to each of various positions (each of sets of coordinates) included in the scan region. Further, on the basis of a histogram of the variance values of the positions, the second calculating function 164 sets a threshold value used for the binarization. For example, on the basis of a discriminant analysis (Otsu's binarization method) by which a threshold value that maximizes a degree of separation is calculated, the second calculating function 164 sets the threshold value indicated with a broken line in FIG. 14. As a result, the second calculating function 164 determines a group of pixels having variance values larger than the threshold value as an artery and determines a group of pixels having variance values smaller than the threshold value as a vein. The vertical axis in FIG. 14 expresses frequency and may express, for example, the number of pixels.

As illustrated in FIG. 15, the image generating circuitry 130 generates the binarized image on the basis of a moment of second or higher order and the threshold value. For example, the image generating circuitry 130 generates the binarized image by assigning, within the scan region, mutually-different pixel values to a group of pixels B1 having variance values larger than the threshold value and to another group of pixels B2 having variance values smaller than the threshold value. In this situation, the group of pixels B1 having the variance values larger than the threshold value corresponds to an artery. In contrast, the group of pixels B2 having the variance values smaller than the threshold value corresponds to a vein.

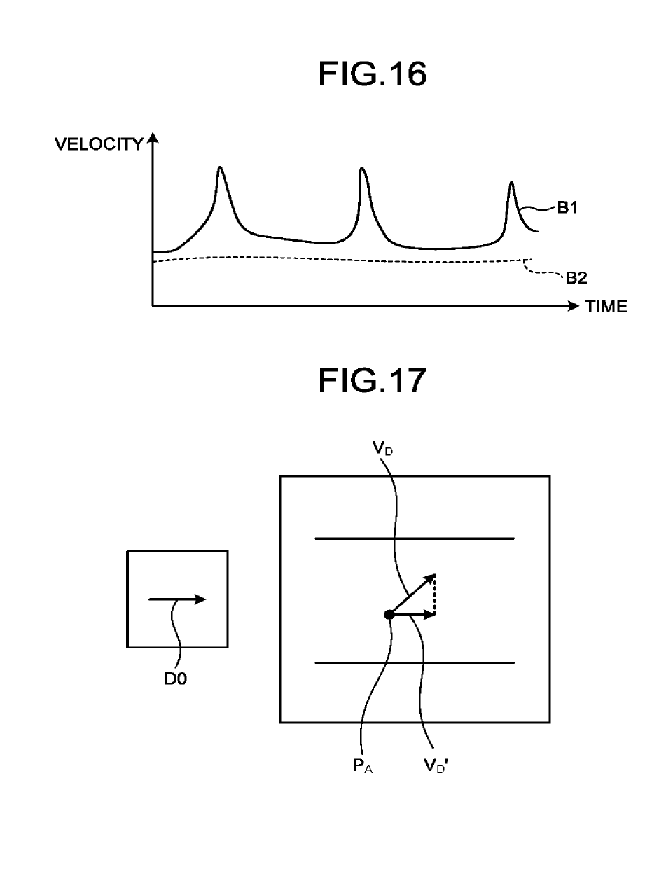

After that, the display controlling function 165 causes the display device 103 to display the binarized image generated by the image generating circuitry 130. In this situation, for example, when displaying a graph (FIG. 16) indicating chronological changes in the velocity of the blood flows in the artery and the vein, the display controlling function 165 is able to display the graph by using mutually-different types of lines (or mutually-different colors) in accordance with the result of the assessment made by the second calculating function 164.

In the manner described above, the ultrasound diagnosis apparatus 1 is capable of presenting the operator with the image in which the artery and the vein are distinguished from each other by binarizing the variance values. In this situation, for other factors besides the variance values, the ultrasound diagnosis apparatus 1 is also able to generate a binarized image by using an n-th order moment.

The illustrations of FIGS. 14 and 15 are merely examples, and possible embodiments are not limited to these examples. For instance, the threshold value does not necessarily have to be set according to Otsu's binarization method and may be set according to any other conventional discriminant analysis method. Further, the threshold value may be set in advance. In that situation, it is desirable to set a threshold value for each imaged site of the body. Further, the threshold value may be set to an arbitrary value by the operator.

### Fourth Embodiment

Further, for example, when the direction of a blood flow in images is known to a certain extent (e.g., in the carotid arteries), the ultrasound diagnosis apparatus 1 is also capable of placing a focus on a projection component of a vector of each bubble toward the blood flow direction that is known and calculating a variance value (an n-th order moment) of the projection component.

In other words, the second calculating function 164 is configured to calculate a moment of second or higher order of the projection component of vectors toward a direction that is set in advance. For example, the second calculating function 164 calculates a projection component of a vector of each bubble toward a direction designated by the operator. After that, the second calculating function 164 calculates a variance value by using the calculated projection component.

FIG. 17 is a drawing for explaining a process performed by the ultrasound diagnosis apparatus 1 according to a fourth embodiment. As illustrated in FIG. 17, the second calculating function 164 sets the direction of an arrow DO, on the basis of an input operation performed by the operator. For example, when the direction of the blood flow in the image is known to a certain extent (e.g., in a carotid artery), the operator designates the blood flow direction by using the input device 102. More specifically, the operator performs an adjusting input operation so as to arrange the direction of the arrow DO displayed on the display device 103 to coincide with the known blood flow direction. In accordance with the input operation, the second calculating function 164 adjusts the direction of the arrow DO.

Subsequently, the second calculating function 164 calculates a projection component of a vector V_{D} in a position PA. For example, the second calculating function 164 calculates a projection component V_{D}′ of the vector V_{D} toward the adjusted direction of the arrow DO. After that, by using the calculated projection component V_{D}′, the second calculating function 164 calculates a variance value (an n-th order moment). Because the process of calculating the variance value is the same as the process explained in the above embodiments, the explanation thereof will be omitted.

In this manner, the ultrasound diagnosis apparatus 1 calculates the moment of second or higher order with respect to the projection component of the vector toward the direction set in advance. With this arrangement, because the ultrasound diagnosis apparatus 1 calculates the variance value by using the vector component in the direction along the blood flow direction, it is possible to more accurately evaluate the dynamics of the blood flow.

### Fifth Embodiment

In the fourth embodiment, the example is explained in which the projection component toward the direction designated by the operator is calculated; however, possible embodiments are not limited to this example. For instance, the ultrasound diagnosis apparatus 1 is also capable of conjecturing a blood flow direction from an image and further calculating a projection component of a vector toward the blood flow direction.

In other words, the specifying function 161 is configured to specify the direction of a tubular site in an image. Further, the second calculating function 164 is configured to calculate a moment of second or higher order of a projection component of a vector toward the specified direction.

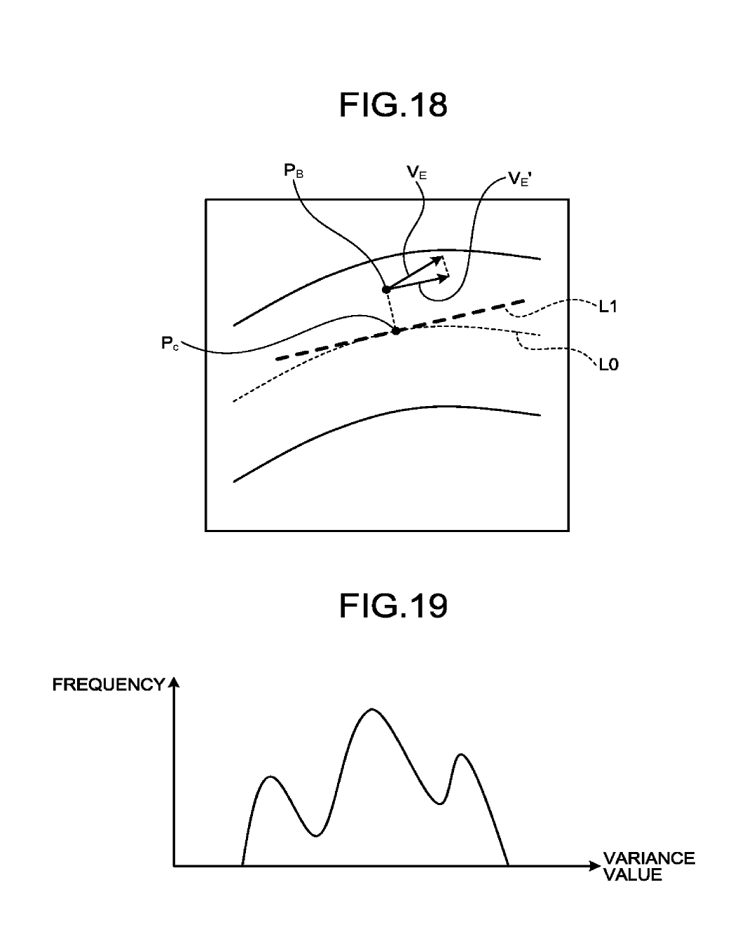

FIG. 18 is a drawing for explaining a process performed by the ultrasound diagnosis apparatus 1 according to a fifth embodiment. As illustrated in FIG. 18, to calculate a vector V_{E} of a bubble in a position P_{B}, the specifying function 161 specifies a central line L0 of the tubular site (the blood vessel) including the position P_{B}. For example, the specifying function 161 detects a region of the tubular site including the position P_{B} from the fundamental image data and specifies the central line L0 of the tubular site by performing an erosion process on the detected region. The process of specifying the central line L0 is not limited by the explanation above. It is possible to specify the central line L0 by using an arbitrary method.

After that, by using the central line L0, the second calculating function 164 calculates a projection component V_{E}′ of the vector V_{E}. For example, the second calculating function 164 specifies a point P_{C} on the central line L0 that is positioned closest to the position P_{B}. Subsequently, the second calculating function 164 specifies a tangential line L1 of the central line L0 that passes through the specified point P_{C}. Further, the second calculating function 164 calculates the projection component V_{E}′ of the vector V_{E} toward the direction of the specified the tangential line L1. After that, the second calculating function 164 calculates a variance value (an n-th order moment) by using the calculated projection component V_{E}′. Because the process of calculating the variance value is the same as the process explained in the above embodiments, the explanation thereof will be omitted.

In the manner described above, the ultrasound diagnosis apparatus 1 is also capable of conjecturing the blood flow direction from the image and further calculating the projection component of the vector toward the blood flow direction. With these arrangements, because the ultrasound diagnosis apparatus 1 calculates the variance value by using the vector component in the direction along the blood flow direction, it is possible to accurately evaluate the dynamics of the blood flows.

### Other Embodiments

Other than the embodiments described above, the present disclosure may be carried out in various different modes.

Displaying a Histogram

Further, for example, the display controlling function 165 is capable of displaying a histogram indicating a distribution of moments of second or higher order, or velocity values, displacements, moving directions, or time periods before arrival of the moving members.

FIG. 19 is a drawing for explaining a process performed by the ultrasound diagnosis apparatus 1 according to another embodiment. In FIG. 19 the horizontal axis expresses variance values. Further, the vertical axis expresses frequency. The frequency may represent the number of pixels in a region of interest set in a single image or may represent the number of pixels in a region of interest set in a plurality of images from the n-th frame to the (n+k)-th frame.

For example, the display controlling function 165 causes the display device 103 to display the histogram illustrated in FIG. 19. In this situation, the horizontal axis and the vertical axis may be changed to represent arbitrary parameters, according to an instruction from the operator. In other words, the display controlling function 165 is configured to display a histogram indicating a distribution of one selected from among: moments of second or higher order; velocity values of the moving members; displacements of the moving members; moving directions of the moving members; and time periods before arrival of the moving members. With these arrangements, the operator is able to visually recognize how much dispersion there is in each of the parameters.

Application to an Optical Ultrasound Diagnosis Apparatus

In the embodiments above, the example is explained in which the dynamics of the blood flows are evaluated by tracking the bubbles; however, possible embodiments are not limited to this example. For instance, it is also possible to render red blood cells in images by using an optical ultrasound diagnosis apparatus and to further evaluate dynamics of blood flows on the basis of tracking of the red blood cells. In this situation, the optical ultrasound diagnosis apparatus is an example of the medical diagnosis apparatus.

For example, the optical ultrasound diagnosis apparatus generates images in a time series in which red blood cells are rendered, by performing a photoacoustic imaging process in a time series, by using substances (the red blood cells) excited by laser light having a wavelength in the range of approximately 400 nm to 700 nm. After that, the optical ultrasound diagnosis apparatus calculates vectors expressing moving of the red blood cells, by arranging the specifying function 161, the setting function 162, and the first calculating function 163 to perform the processes on the images in the time series in which the red blood cells are rendered. Subsequently, the optical ultrasound diagnosis apparatus calculates a moment of second or higher order (e.g., a variance value) related to movement information of the moving members (the red blood cells) by arranging the second calculating function 164 to perform the process while using the vectors expressing the moving of the red blood cells. With these arrangements, the optical ultrasound diagnosis apparatus is able to accurately evaluate the dynamics of the blood flows, similarly to the ultrasound diagnosis apparatus 1 according to the embodiments described above.

It is also possible to render red blood cells in images by transmitting and receiving an ultrasound wave having a radio frequency. Accordingly, the process of tracking the red blood cells described above may be performed not only by the optical ultrasound diagnosis apparatus, but also by the ultrasound diagnosis apparatus 1.

Other Medical Diagnosis Apparatuses

Further, the processing functions according to any of the embodiments described above is applicable, not only to the ultrasound diagnosis apparatus 1 and to the optical ultrasound diagnosis apparatus, but also to other medical diagnosis apparatuses as long as the medical diagnosis apparatuses are capable of rendering moving members in images. For example, examples of the other medical diagnosis apparatuses that are applicable include X-ray diagnosis apparatuses, X-ray Computed Tomography (CT) apparatuses, Magnetic Resonance Imaging (MRI) apparatuses, Single Photon Emission Computed Tomography (SPECT) apparatuses, Positron Emission computed Tomography (PET) apparatuses, SPECT-CT apparatuses in each of which a SPECT apparatus and an X-RAY CT apparatus are integrally combined, PET-CT apparatuses in each of which a PET apparatus and an X-ray CT apparatus are integrally combined, or a group of apparatuses made up of any of these.

Medical Image Processing Apparatuses

Further, in the embodiments described above, the example is explained in which the ultrasound diagnosis apparatus 1 is configured to take the ultrasound image data and to perform the processes in the embodiments by using the ultrasound image data taken; however, possible embodiments are not limited to this example. For instance, the processes in the embodiments are also applicable to a medical image processing apparatus having no imaging function.

For example, the medical image processing apparatus is configured to obtain ultrasound image data that has already been taken, from an apparatus such as the ultrasound diagnosis apparatus 1 or a medical image storage device. In other words, the medical image processing apparatus has an obtaining function to obtain images in a time series, on the basis of a result of a scan performed on a scan region. The obtaining function is installed, for example, in processing circuitry provided on the inside of the medical image processing apparatus. In this situation, the obtaining function is an example of the obtaining unit.

Further, the medical image processing apparatus performs the processes according to any of the embodiments described above on the obtained ultrasound image data. In other words, the processing circuitry included in the medical image processing apparatus has the same functions as those of the processing circuitry 160 described above. For example, the processing circuitry included in the medical image processing apparatus includes a specifying function, a first calculating function, and a second calculating function. The specifying function, the first calculating function, and the second calculating function perform the same processes as those performed by the specifying function 161, the first calculating function 163, and the second calculating function 164, respectively. With these arrangements, it is possible to apply the processes according to any of the embodiments described above to the medical image processing apparatus.

Using a Moment of First Order

Further, for example, in the embodiments described above, the example is explained in which the moment of second or higher order is calculated; however, possible embodiments are not limited to this example. For instance, as indicated in Expression (7) presented below, the second calculating function 164 is also capable of calculating a first order moment. It is possible to use the first order moment as an index value indicating dispersion or spread of certain values, similarly to a moment of second or higher order.

$$σ = \frac{1}{N} {\int }_{t = s}^{e} \vert v ( t ) - μ \vert dt = \frac{1}{N} {\sum }_{t = s}^{e} \vert v ( t ) - μ \vert dt \qquad ( 7 )$$

In Expression (7), σ denotes the first order moment (which may be referred to as a standard deviation). Because the explanations of V(t), t, μ, N, s, and e are the same as those in the above embodiments, the explanations thereof will be omitted.

In other words, the second calculating function 164 serving as an obtaining unit is configured to obtain the moment of first or higher order related to the movement information of the moving members, with respect to at least a part of the scan region. With these arrangements, the ultrasound diagnosis apparatus 1 is able to accurately evaluate the dynamics of the blood flows.

Further, the constituent elements of the apparatuses and the devices illustrated in the drawings are based on functional concepts. Thus, it is not necessary to physically configure the constituent elements as indicated in the drawings. In other words, the specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. For example, the functions of the image generating circuitry 130 described above may be integrated with the functions of the processing circuitry 160. Further, all or an arbitrary part of the processing functions performed by the apparatuses and the devices may be realized by a CPU and a program that is analyzed and executed by the CPU or may be realized as hardware using wired logic.

Further, with regard to the processes explained in the embodiments above, it is acceptable to manually perform all or a part of the processes described as being performed automatically. Conversely, by using a method that is publicly known, it is also acceptable to automatically perform all or a part of the processes described as being performed manually. Further, unless noted otherwise, it is acceptable to arbitrarily modify any of the processing procedures, the controlling procedures, specific names, and various information including various types of data and parameters that are presented in the above text and the drawings.

Further, the image processing methods explained in the above embodiments may be realized by causing a computer such as a personal computer or a workstation to execute an image processing program prepared in advance. The image processing program may be distributed via a network such as the Internet. Further, the image processing program may be recorded on a computer-readable recording medium such as a hard disk, a flexible disk (FD), Compact Disk Read-Only Memory (CD-ROM), a Magneto-Optical (MO) disk, or a Digital Versatile Disk (DVD), so as to be executed as being read from the recording medium by a computer.

In the embodiments and the modification examples described above, the expression “real-time” means performing a process immediately every time a piece of data serving as a processing target is generated. For example, the process of displaying an image in a real-time manner does not necessarily require that the time at which a patient is imaged exactly coincides with the time at which the image is displayed. The image may be displayed with a slight delay caused by the time period required by processes such as an image processing process.

Further, in the embodiments and modification examples described above, the word “images” does not refer only to the images displayed on the display device 103. For example, the word “images” refers to a concept including image data in which each of the pixel positions included in each of the images is kept in correspondence with a pixel value in the pixel position.

According to at least one aspect of the embodiments described above, it is possible to accurately evaluate the dynamics of the blood flows.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical diagnosis apparatus comprising processing circuitry configured:
to generate images in a time series on a basis of a result of a scan performed on a scan region;
to specify a position of a moving member included in the scan region, with respect to each of the images in the time series;
to obtain movement information of the moving member on a basis of the positions of the moving member; and
to obtain a moment of first or higher order related to the movement information of the moving member, with respect to at least a part of the scan region.

2. The medical diagnosis apparatus according to claim 1, wherein
as the movement information, the processing circuitry calculates one selected from among velocity, a displacement, a moving direction, and a time period before arrival, with respect to the moving member, and
the processing circuitry calculates the moment of first or higher order around one selected from among an average value, a median value, and an origin, with respect to the movement information.

3. The medical diagnosis apparatus according to claim 2, wherein, as the moment of first or higher order, the processing circuitry calculates a variable value that is temporal, spatial, or spatiotemporal.

4. The medical diagnosis apparatus according to claim 1, wherein
the processing circuitry calculates the moment of first or higher order with respect to each of various positions in a region of interest within the scan region, and
the processing circuitry further generates a second image structured with pixels each having a pixel value expressing the moment of first or higher order.

5. The medical diagnosis apparatus according to claim 1, wherein the processing circuitry generates a binarized image on a basis of the moment of first or higher order and a threshold value.

6. The medical diagnosis apparatus according to claim 1, wherein
as the movement information, the processing circuitry calculates a vector of the moving member, and
the processing circuitry calculates the moment of first or higher order of a projection component of the vector toward a direction set in advance.

7. The medical diagnosis apparatus according to claim 1, wherein
as the movement information, the processing circuitry calculates a vector of the moving member,
the processing circuitry specifies a direction of a tubular site in each of the images, and
the processing circuitry calculates the moment of first or higher order of a projection component of the vector toward the direction.

8. The medical diagnosis apparatus according to claim 1, wherein the processing circuitry displays a histogram indicating a distribution of one selected from among: values of the moment of first or higher order; velocity values of the moving member; displacements of the moving member; moving directions of the moving member; and time periods before arrival of the moving member.

9. The medical diagnosis apparatus according to claim 1, wherein the medical diagnosis apparatus is an ultrasound diagnosis apparatus.

10. The medical diagnosis apparatus according to claim 9, wherein the movement information includes a component in a direction different from a direction of an ultrasound scan performed on the scan region.

11. The medical diagnosis apparatus according to claim 9, wherein the moving member is a bubble.

12. The medical diagnosis apparatus according to claim 11, wherein
the processing circuitry specifies the position of the bubble with respect to each of the images in the time series, and
the processing circuitry calculates a vector expressing moving of the bubble, by tracking the position of the bubble in each of the images in the time series.

13. A medical image processing apparatus comprising processing circuitry configured:
to generate images in a time series on a basis of a result of a scan performed on a scan region;
to specify a position of a moving member included in the scan region, with respect to each of the images in the time series;
to obtain movement information of the moving member on a basis of the positions of the moving member; and
to obtain a moment of first or higher order related to the movement information of the moving member, with respect to at least a part of the scan region.

14. An image processing method comprising:
obtaining images in a time series on a basis of a result of a scan performed on a scan region;
specifying a position of a moving member included in the scan region, with respect to each of the images in the time series;
obtaining movement information of the moving member on a basis of the positions of the moving member; and
obtaining a moment of first or higher order related to the movement information of the moving member, with respect to at least a part of the scan region.
